(19) United States
(12) Patent Application Publication
(10) Pub. No.: US 2019/0124479 A1
(43) Pub. Date: Apr. 25, 2019

(54) CLOUD-BASED CLINICAL LOGGING

(71) Applicant: GoodDoc, LLC, Hockessin, DE (US)

(72) Inventors: Raghav Garg, Hockessin, DE (US); Manish Garg, Hockessin, DE (US)

(21) Appl. No.: 16/165,853

(22) Filed: Oct. 19, 2018

Related U.S. Application Data
(60) Provisional application No. 62/574,447, filed on Oct. 19, 2017.

(51) Int. Cl. H04W 4/12; G16H 40/40; G16H 40/60; H04L 12/26; G06F 15/18

(52) U.S. Cl. CPC H04W 4/12; G16H 40/40; G06F 15/18; H04L 43/04; G16H 40/60

(57) ABSTRACT

Cloud-based clinical logging: A method may include obtaining from a first medical device, by a processor, a first datum at a first time; recording the first datum in a database; and transmitting a first message to a first person regarding the first datum if the first datum does not satisfy a pre-determined threshold, where the first message requests an action based on the first datum not satisfying the pre-determined threshold.

### PRIORITY

This application claims priority to U.S. Provisional Patent Application No. 62/574,447, filed Oct. 19, 2017, the disclosure of which is incorporated by reference herein.

### BACKGROUND

Many government agencies, such as the United States Department of Health, establish procedures and regulations for medical clinics. For example, dialysis clinics may be mandated to monitor a plurality of parameters necessary to ensure patient safety and keep a plurality of logs. Parameters may include data related to water purity and quality, temperature, chemical makeup, and the parameters may need to be checked multiple times a day.

Currently, the logs are maintained by hand and may be maintained by multiple different staff members (e.g., nurses, technicians, managers). As such, maintaining the logs may be cumbersome, prone to inaccuracy or falsehood, and because they are frequently handwritten, may be illegible. In addition, due to the number of records maintained, the log registers may be large and difficult to review and/or analyze.

In some cases, technicians logging data may enter a repeated value for multiple entries without determining the actual parameter value determined at a given time. In some cases, the timing of obtaining a value may be important, but a technician may be late or may miss a collection time; for example, values may need to be collected at 9:00 a.m., 12:00 p.m., 3:00 p.m., etc., but a technician may collect values at 9:30 a.m., miss the 12:00 p.m. collection, and collect the 3:00 p.m. value at 4:30 p.m. In such cases, the technician may simply write down values for 9:00 a.m., 12:00 p.m., and 3:00 p.m., even though those may not be the actual values of the various parameters at those times. Hence, the records may be falsified. In additional or alternative cases, two-person verification may be required on entry of a value, however, a second person's signature may be forged or may not be obtained at all. Thus, the maintenance of paper logs and potential human error results in logs not being maintained properly or with the requisite accuracy to ensure patient safety. Frequently, the lack of proper maintenance also results in a lack of compliance of the regulations set forth by agencies such as the United States Department of Health.

The subject matter claimed herein is not limited to embodiments that solve any disadvantages or that operate only in environments such as those described above. Rather, this background is only provided to illustrate one example technology area where some embodiments described herein may be practiced.

### SUMMARY

In one embodiment, a computer-implemented method for cloud-based clinical logging may include obtaining from a first medical device, by a processor, first datum at a first time; recording the first datum in a database; and transmitting a first message to a first person regarding the first datum if the first datum does not satisfy a pre-determined threshold, where the first message requests an action based on the first datum not satisfying the pre-determined threshold.

In one embodiment, transmitting the first message may further include transmitting a text message, e-mail, telephone call, instant message, or a combination thereof.

In one embodiment, the method may further include initiating an audio alarm, a visual alarm, or a combination thereof based on the first datum not satisfying the pre-determined threshold.

In one embodiment, the method may further include determining that the requested action is not satisfied; and transmitting a second message to a second person regarding the first message and the first datum based on determining that the requested action is not satisfied. In one embodiment, determining that the first datum does not satisfy the pre-determined threshold may further include obtaining a range of acceptable values for the first datum; and determining that a value associated with the first datum is outside of the range of acceptable values.

In one embodiment, the method may include determining a requested frequency for obtaining the first datum and at least a subsequent datum; obtaining the subsequent datum based on the requested frequency; and transmitting a third message to the first person regarding the subsequent datum if the subsequent datum does not satisfy the pre-determined threshold.

In one embodiment, the method may include determining a time range between obtaining the first datum and at least a subsequent datum; determining that the subsequent datum is not obtained within the time range; and transmitting a fourth message to the first person regarding the subsequent datum if the subsequent datum is not obtained within the time range.

The method may include obtaining from the medical device, a second datum at a second time; recording the second datum in the database; determining a trend between the first datum and the second datum; predicting failure of a component of the medical device based on the determined trend; and requesting a second action based on predicting failure of the component. In one embodiment, predicting failure of the component may further include training a machine learning model on at least historical data obtained from the medical device, the first datum, and the second datum.

In some embodiments, a computer device may include a processor, a memory in electronic communication with the processor, and instructions stored in the memory, with the instructions being executable by the processor to perform a method for cloud-based clinical logging.

In some embodiments, one or more non-transitory computer-readable media may include one or more computer-readable instructions that, when executed by one or more computing devices, cause the one or more computing devices to perform a method for cloud-based clinical logging.

It is to be understood that both the foregoing summary and the following detailed description are explanatory and are not restrictive of the invention as claimed.

### BRIEF DESCRIPTION OF THE DRAWINGS

Embodiments will be described and explained with additional specificity and detail through the use of the accompanying drawings in which:

FIG. 1 illustrates an example system that may be used for cloud-based clinical logging, in accordance with at least one embodiment;

FIG. 2 illustrates a block diagram of a device related to cloud-based clinical logging, in accordance with at least one embodiment;

FIGS. 3A-3E illustrate example interfaces related to cloud-based clinical logging, in accordance with at least one embodiment;

FIGS. 4A-4G illustrate example interfaces related to cloud-based clinical logging, in accordance with at least one embodiment;

FIGS. 5A-5H illustrate example interfaces related to cloud-based clinical logging, in accordance with at least one embodiment;

FIG. 6 illustrates a flowchart of an example method related to cloud-based clinical logging, in accordance with at least one embodiment; and

FIG. 7 illustrates an example computer system for cloud-based clinical logging, in accordance with at least one embodiment.

### DETAILED DESCRIPTION

Paper logs are prone to errors, both in the accurate collection of data and in the accurate recording of data. In some cases, data are used to determine whether certain aspects of patient care are sufficient safe for a patient, including whether or not treatment should be started, should be stopped, if attention is needed with regard to an element of the treatment (e.g., whether water in a dialysis machine is sufficiently clean), and/or whether or not attention is needed with regard to a component of a machine. In some cases, data gathering and analysis may be done on a microcosmic scale (e.g., at a single location, a single clinic, a single city), which may not be using best practices when compared to the data gathered and analysis done on a macrocosmic scale (e.g., multiple locations, clinics in a state, United States clinics). Thus, it is important to provide methods and/or systems that enable accurate, data gathering, reporting, and analysis in order to meet regulations and to provide increased and consistent patient safety. In one embodiment, data collection and analysis may be done automatically and digitally at a plurality of sensors, where the data may be accessible to a number of users, and stored in a secure manner.

Some embodiments disclosed herein may enable cloud-based clinical logging. In one example embodiment, a person may be a patient at a dialysis clinic. In some cases, part of the patient's treatment may include hemodialysis, which uses a predetermined amount of water for the production of dialysis fluids. Due to the nature of the procedure, the water used in hemodialysis may come in direct contact with the patient's blood, and thus the characteristics of the water are important for the patient's safety, including the purity, the temperature, the volume, the flow, and other parameters.

In some embodiments, a technician may be tasked with reading one of many potential analog or digital gauges associated with a plurality of machines and devices. Once the technician reads the value, the technician may then enter the data obtained into a paper document; however, the entries may be time-sensitive, may need to be specific to a certain decimal point, may need two-party authentication, etc., and as such, may be prone to human error. A cloud-based clinical logging solution is presented.

Although the descriptions herein are provided with respect to dialysis treatment, the methods and systems are not limited to dialysis treatment and may apply to other types of medical treatment and to other industries, including, but not limited to medication storage, crash cart monitoring, other healthcare industries, transportation, shipping, aerospace, aeronautics, oil and gas exploration, law enforcement, military, education, and the like.

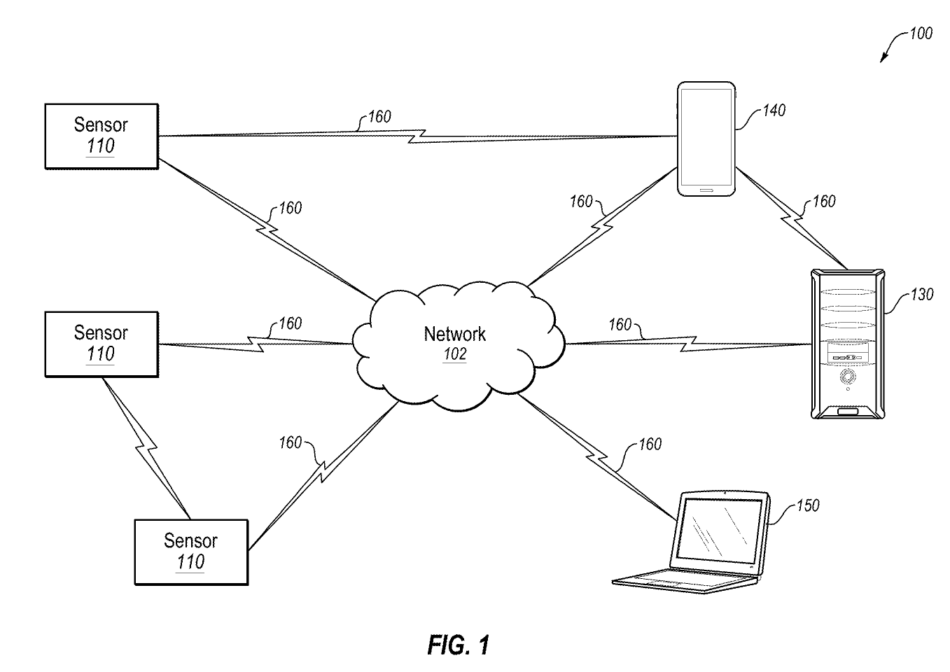

Turning to the figures, FIG. 1 illustrates an example system 100 that may be used for cloud-based clinical logging, in accordance with at least one embodiment. The system 100 may include one or more sensors 110, network 120, server 130, local computing device 140 and/or remote computing device 150. The network 120 may communicate by way of wired or wireless communication links 160 with sensors 110, local computing device 140, remote computing device 150, and server 130.

The computing devices 140 and 150 may be dispersed throughout the system 100 and each device 140 and 150, respectively, may be stationary and/or mobile. Computing devices 140 and 150 may include, but are not limited to, a cellular phone, a tablet computer, a wearable electronic device (e.g., a smart watch, a biometric sensor and/or tracker), a personal digital assistant (PDA), a wireless modem, a handheld device, a laptop computer, a cordless phone, a wireless local loop (WLL) station, a display device (e.g., a television set, a computer monitor, etc.), a printer, a camera, or any combination of the above. Computing devices 140 and 150 may also include or be referred to by those skilled in the art as a user device, a user equipment, a smartphone, a BLUETOOTH® device, a Wi-Fi device, a mobile station, a subscriber station, a remote unit, a handset, a user agent, a mobile client, a client, and/or some other suitable terminology.

The computing devices 140 and 150, as well as the server 130, may wirelessly communicate with the sensors 110 via one or more antennas. In additional or alternative embodiments, computing devices 140 and 150, as well as the server 130 may communicate via wired communication with the sensors 110. The sensors 110 may be dispersed throughout the system 100 and each sensor 110 may be stationary and/or mobile. A sensor 110 may include and/or be one or more sensors that determine values related to, but not limited to: dates, times, water temperature, refrigeration temperature, crash cart checks, machine numbers, serial numbers, disinfection values, acidity, corrective actions, amount of ice present in a freezer, citric acid mixing, amount of bleach, lot numbers, expiration dates, volume of liquids, weight of solids, and the like. In some embodiments, the sensors 110 may be analog gauges. In additional or alternative embodiments, sensors 110 may be digital sensors.

Computing devices 140 and/or 150, server 130, and/or at least one sensor 110 may communicate through one or more wired and/or wireless connections with various components such as medical equipment, control panels, antennas, base stations, and/or network equipment (e.g., other servers, wireless communication points, etc.).

The communication links 160 illustrated in system 100 may include uplink (UL) transmissions from devices 140 and 150 and/or sensors 110 to other devices in the system 100, and/or downlink (DL) transmissions, from devices within system 100 to devices 140 and/or 150 and/or server 130. The downlink transmissions may also be called forward link transmissions while the uplink transmissions may also be called reverse link transmissions. Each communication link 160 may include one or more carriers, where each carrier may be a signal made up of multiple sub-carriers (e.g., waveform signals of different frequencies) modulated according to the various radio technologies. Each modulated signal may be sent on a different sub-carrier and may carry control information (e.g., reference signals, control channels, etc.), overhead information, user data, etc. The communications links 160 may transmit bidirectional communications and/or unidirectional communications, including, but not limited to 345 MHz, Wi-Fi, BLUETOOTH®, BLUETOOTH® Low Energy, cellular, Z-WAVE®, millimeter wave, 802.11, peer-to-peer (P2P), LAN, WLAN, Ethernet, firewire, fiber optic, and/or other connection types.

In some embodiments, computing devices 140 and/or 150 may include one or more antennas configured to employ antenna diversity schemes to improve communication quality and reliability between the control panel and the computing devices. Additionally or alternatively, devices 140 and 150 may employ multiple-input, multiple-output (MIMO) techniques that may take advantage of multi-path, mesh-type environments to transmit multiple spatial layers carrying the same or different coded data.

While the computing devices 140 and/or 150 and/or server 130 may communicate with each other using communication links 160, each computing device 130, 140, and/or 150 may communicate with one or more other devices by way of one or more direct communications links 160. Two or more computing devices 130, 140, and/or 150 communicate via a direct communication link 160 when computing devices 130, 140, and 150 are in the same geographic coverage area or when one or none of the computing devices 130, 140 or 150 is within the geographic coverage area. Examples of direct communication links 160 may include Wi-Fi Direct, BLUETOOH®, wired, and/or other P2P group connections. The computing devices 130, 140, and 150 in these examples may communicate according to the Wide Local Area Network (WLAN) radio and baseband protocol including physical and Media Access Control (MAC) layers from the Institute of Electrical and Electronics Engineers (IEEE) standards, such as 802.11, and its various versions including, but not limited to: 801.11a, 802.11b, 802.11g, 802.11n, 802.11ac, 802.11ad, and 802.11ah. In other implementations, other P2P connections and/or ad hoc networks may be implemented within system 100. In additional sensors 110 may communicate with devices 130, 140, and/or 150 through direct links 160 or through the network 120.

In some examples, local computing device 140 may be a custom computing entity configured to interact with sensors 110 via network 120 and/or server 130. In other examples, local computing device 140 may be a general purpose computing entity such as a personal computing device; for example, local computing device 140 may be a desktop computer, a laptop computer, a netbook, a tablet personal computer (PC), a control panel, an indicator panel, a multi-site dashboard, an IPOD®, a smartphone, a cellular phone, a PDA, and/or any other suitable device operable to send and receive signals, store and retrieve data, and/or execute software components.

The computing devices 140 and 150 may include memory, a processor, an output, a data input, and a communications component. The processor may be, but is not limited to, a general purpose processor, a Field Programmable Gate Array (FPGA), an Application Specific Integrated Circuit (ASIC), a Digital Signal Processor (DSP), etc. The processor may be configured to retrieve data from and/or write data to the memory.

The memory may be, but is not limited to, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, or cloud storage. In some embodiments, the computing devices 140 and 150 may include one or more hardware-based modules (e.g., FPGA, ASIC, DSP, etc.) and/or software-based modules (e.g., a module of computer code stored at the memory and executed at the processor, a set of processor-readable instructions that may be stored at the memory and executed at the processor) associated with executing an application, such as, for example, receiving, analyzing, transmitting, and/or displaying data from the sensors 110.

The processor of the local computing device 140 may be operable to control operation of the output of the local computing device 140. The output may be a television, a liquid crystal display (LCD) monitor, a cathode ray tube (CRT) monitor, plasma monitor, speaker, tactile output devices (e.g., a capacitive touchscreen device), smart speaker, audio devices, holograms, etc. In some embodiments, the output may be an integral component of the local computing devices 140; for example, the output may be directly coupled to the processor. In some embodiments, an output component may include, but is not limited to, a High Definition Multimedia Interface™ (HDMI) connector, a Video Graphics Array (VGA) connector, a Universal Serial Bus™ (USB) connector, a tip, ring, sleeve (TRS) connectors, and/or any other suitable connector operable to couple the local computing device 140 to the output.

The remote computing device 150 may be a computing entity operable to enable a remote user to monitor the output of the sensors 110 and/or the local computing device 140. The remote computing device 150 may be functionally and/or structurally similar to the local computing device 140 and may be operable to receive data streams from and/or send signals to at least one of the sensors 110 via the network 120. The network 120 may be the Internet, an intranet, a personal area network PAN, a local area network (LAN), a wide area network (WAN), a virtual network, a telecommunications network implemented as a wired network and/or wireless network, etc. The remote computing devices 150 may receive and/or send signals over the network 120 by way of communication links 160 and server 130.

In some embodiments, one or more sensors 110 may communicate through wired and/or wireless communication links 160 with one or more of the computing devices 140 and 150, and the network 120. The network 120 may communicate through wired and/or wireless communication links 160 with the computing devices 140 and 150 through server 130. In another embodiment, the network 120 may be integrated with any of the computing devices 140 and 150 and/or server 140 such that separate components are not required. Additionally, in another embodiment, one or more sensors 110 may be integrated with the local computing device 140 such that separate components may not be needed.

In some embodiments, the one or more sensors 110 may be sensors configured to conduct periodic or ongoing automatic measurements related to dates, times, water temperature, refrigeration temperature, crash cart checks, machine numbers, serial numbers, disinfection, acidity, corrective actions, amount of ice present in a freezer, citric acid mixing, amount of bleach, lot numbers, expiration dates, volume of liquids, weight of solids, identification of a technician (including biometrics, personal identification numbers, passwords, etc.) and the like. Each sensor 110 may be enabled to determine multiple parameters, or alternatively, separate sensor units 110 may monitor separate parameters.

In some embodiments, the parameter data communicated to the local and/or remote computing devices 140 and 150 may be encrypted.

In some embodiments, the one or more sensors 110 may be positioned at various locations throughout a property (e.g., a hospital, a clinic, a house, an apartment, a room, an office building, outside of a building, etc.). In other embodiments, the one or more sensors 110 may be integrated or collocated with other devices and/or machines (e.g., medical devices, filters) building system components, appliances, building fixtures, and the like. In any embodiment, each of the one or more sensors 110, and/or local computing device 140 may include a speaker unit, a microphone unit, and/or a camera unit.

The server 130 may be configured to communicate with the one or more sensor 110, local computing device 140, and remote computing device 150. The server may perform additional processing on signals received from the one or more sensors 110 and/or local computing device 140, transmit the received information to the remote computing device 150 and/or sensor 130.

Server 130 may be a computing device operable to receive data streams, store, and/or process data, and/or transmit data and/or data summaries and analysis. The server 130 may include a database (e.g., in memory) containing parameters and analysis data received, determined and/or obtained from the sensors 110 and/or the local computing device 140. Additionally, software (e.g., stored in memory) may be executed on a processor of the server 130.

Modifications, additions, or omissions may be made to FIG. 1 without departing from the scope of the present disclosure. For example, the system 100 may include more or fewer elements than those illustrated and described in the present disclosure. In addition, in some embodiments, sensors 110, local and/or remote computing devices 140 and 150, respectively, and/or server 130 may be combined such that they may be considered the same device.

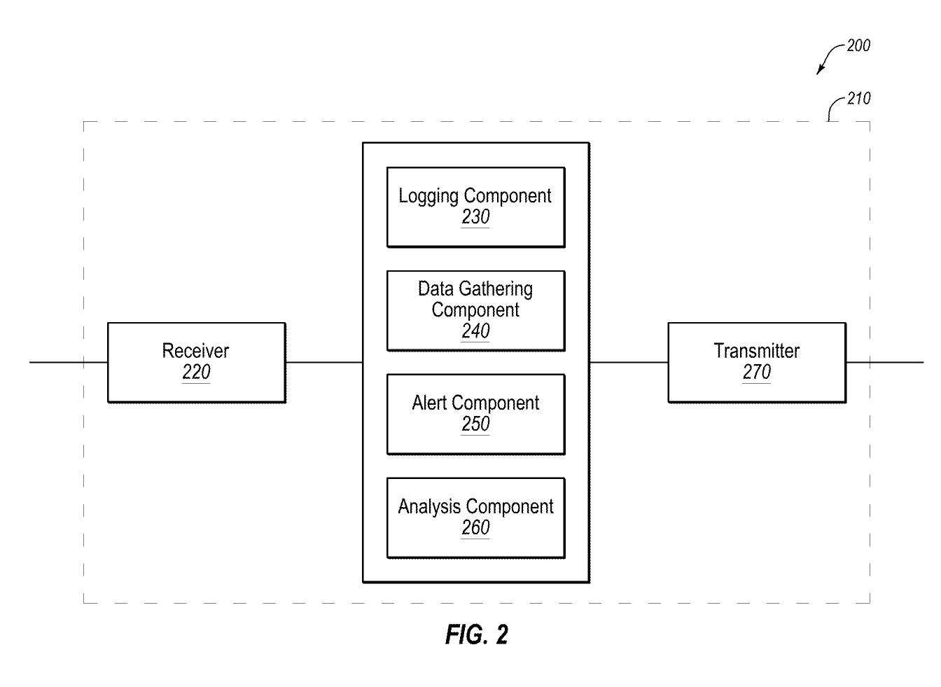

FIG. 2 illustrates a block diagram of an example apparatus 210 for use in cloud-based clinical logging, in accordance with at least one embodiment. The apparatus 210 may be an example of one or more aspects of local computing device 140 and/or remote computing device 150 and/or any of the sensors 110. The apparatus 210 may include a receiver 220, a logging component 230, and/or a transmitter 270. The apparatus 210 may also be or include a processor. Each of the components 220, 230, 240, 250, 260 and/or 270 may be in communication with one another—directly and/or indirectly.

The components of the apparatus 210 may, individually or collectively, be implemented using one or more application specific integrated circuits (ASICs) adapted to perform some or all of the applicable functions in hardware. Alternatively, the functions may be performed by one or more other processing units or cores, on one or more integrated circuits. In other examples, other types of integrated circuits may be used (e.g., Structure/Platform ASICs, Field Programmable Gate Arrays (FPGAs), and other Semi-Custom ICs), which may be programmed in any manner known in the art. The functions of each component may also be implemented—in whole or in part—with instructions embodied in memory formatted to be executed by one or more general and/or application-specific processors.

The receiver 220 may receive information such as packets, user data, and/or control information associated with various information channels (e.g., control channels, data channels, etc.). The receiver 220 may be configured to receive data, audio and/or video streams from the sensors 110, local computing device 140, and/or remote computing device 150. Received data, may be passed onto logging component 230. In addition, the logging component 230 itself may detect data, audio, video, location, time, and/or verification information at the apparatus 210 and may communicate the data to a transmitter 270, and/or to other components of the apparatus 210 not specifically illustrated in FIG. 2.

The transmitter 270 may then communicate the data to the remote computing device 150, the server 130, and/or a third-party. Logging component 230 may further include any of a data gathering component 240, an alert component 250, and analysis component 260. In some embodiments, logging component 230 may enable the secure collection of timely, accurate, legible, and accessible data. Logging component 230 may operate as a software application running on, for example, a local computing device 140. In some embodiments, data collection may be done manually by a technician (i.e., technician looks at a gauge and manually enters data into the logging component), whereas in additional or alternative embodiments, data collection may be automatic (i.e., sensors 110 may automatically determine data values and send them to local computing device 140, remote computing device 150, and/or server 130.) In some embodiments, the software application may automatically request and/or receive data from sensors 110 or from other computing devices.

In some embodiments, data gathering component 240 may obtain and/or determine data. In an example embodiment, patients at a dialysis clinic may receive hemodialysis treatment which involves cleaning the patient's blood using a dialysis machine. Although dialysis is used as an example embodiment, many other medical treatments as well as other industries may benefit from the methods and systems described herein.

In one embodiment, the hemodialysis treatment (or other treatment), may use water. Water may be obtained from an outside source, such as a municipal reservoir (e.g., tap water). Tap water, however, may not be pure or clean enough for medical treatment. Furthermore, the chemical makeup and temperature of water received directly from an outside source may not be safe or sufficient. In additional or alternative embodiments, impurities such as animals, bugs, chemicals, soil, etc. may be in the water. In additional or alternative embodiments, the water may further be too cold, too hot, too basic, too acidic, etc. Thus, in order to prepare the water for use in medical treatment, the water may be passed through one or more various filters, altered, heated or cooled, etc., before being used in treatment.

In one embodiment, a technician may be tasked with a schedule to check the values of certain parameters at a plurality of machines throughout the day. The technician may be a nurse, doctor, intern, or any person having permission to access the machinery. The schedule may include multiple checks of a single machine at various times through a day, a week, a month, etc., or may include multiple checks of multiple machines at multiple times. For example, a single machine may be checked every four hours and may have a single value, whereas in other embodiments, a single machine may be checked every day and may have 35 values. The number of machines, checks, and data may be any number.

Furthermore, multiple machines and devices are likely used in preparing the water for patient use, and thus, there may be multiple stages at which data related to the water may be obtained from when the water is received at the clinic until the water is used in treatment.

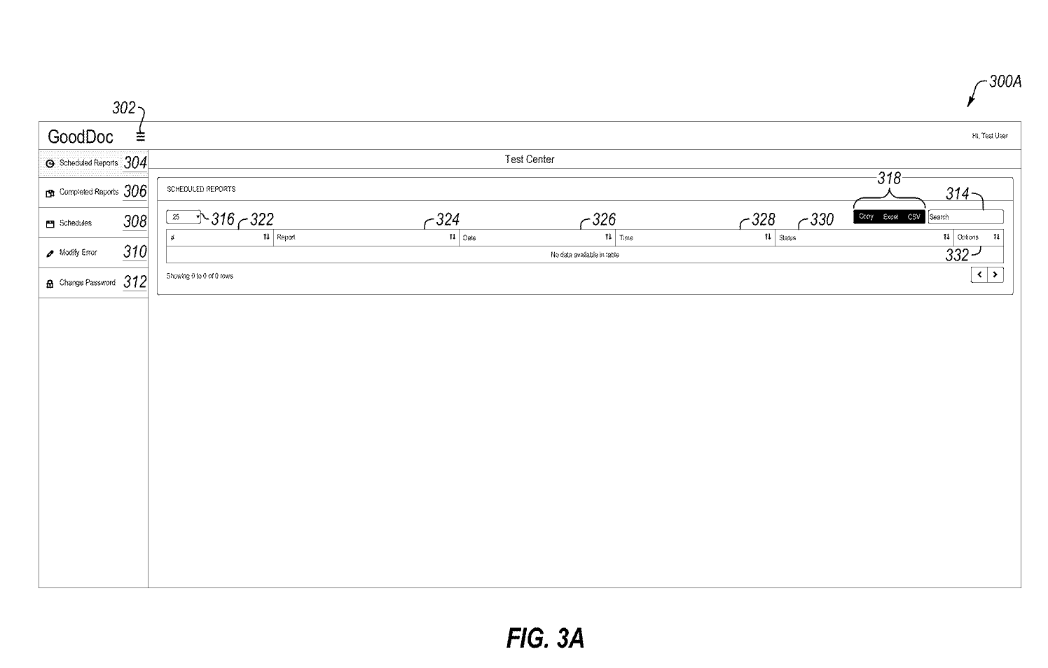

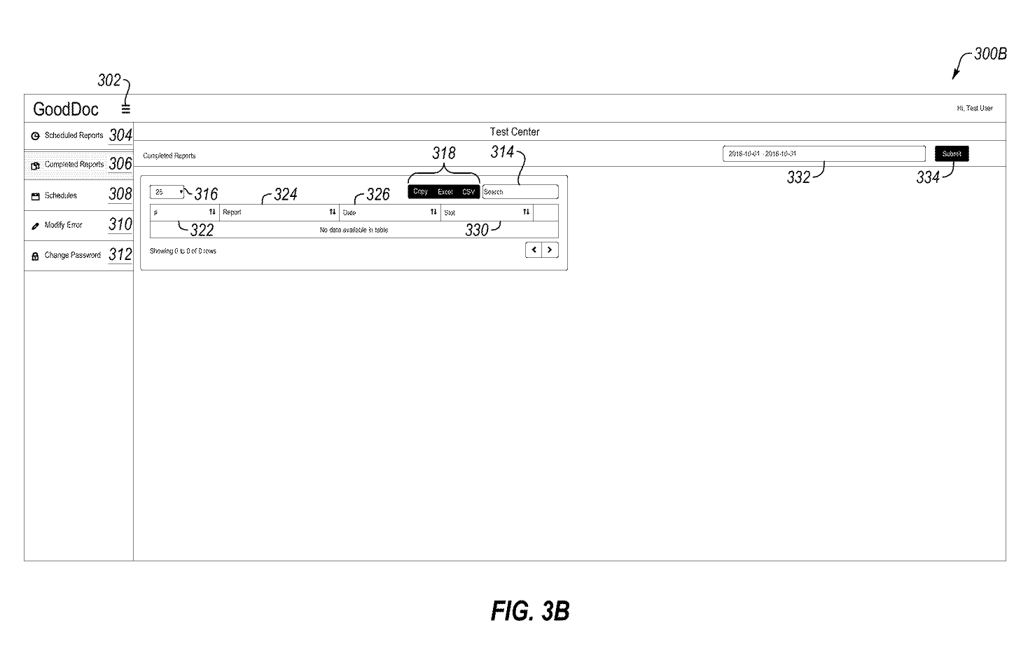

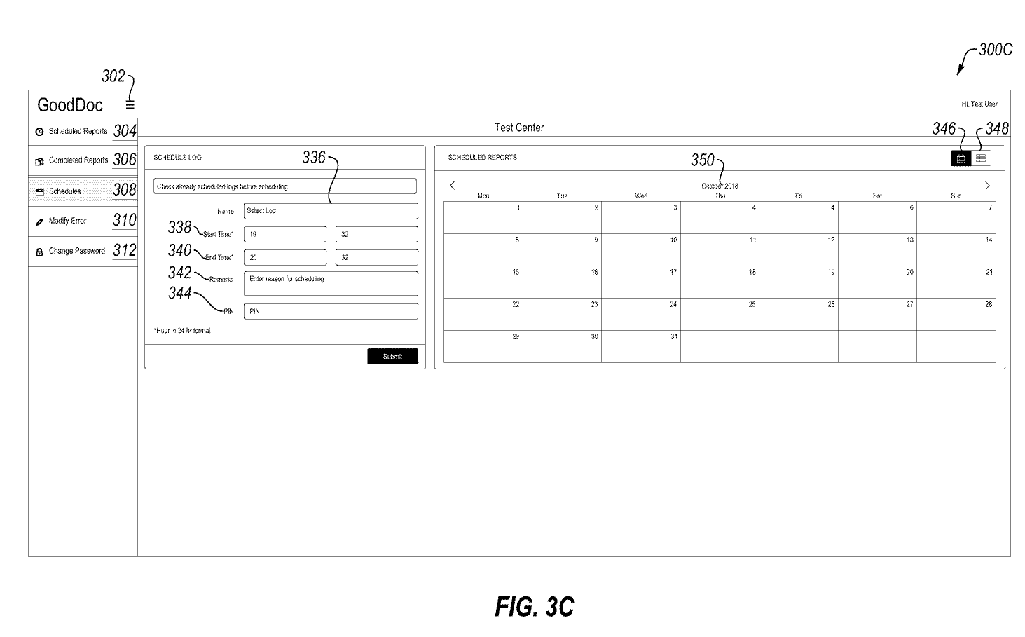

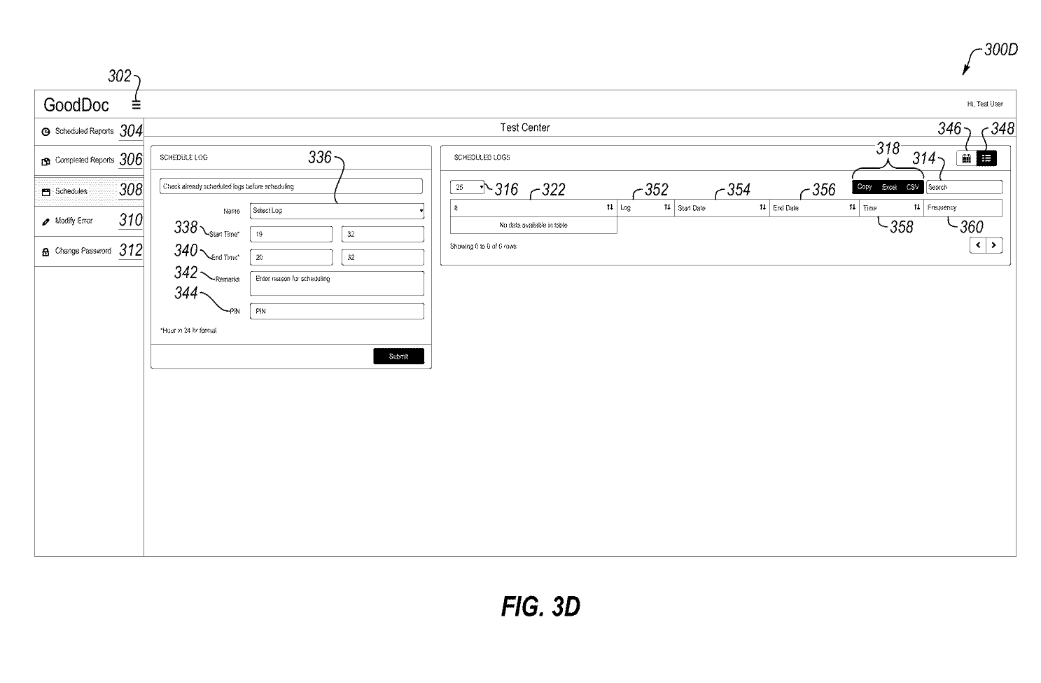

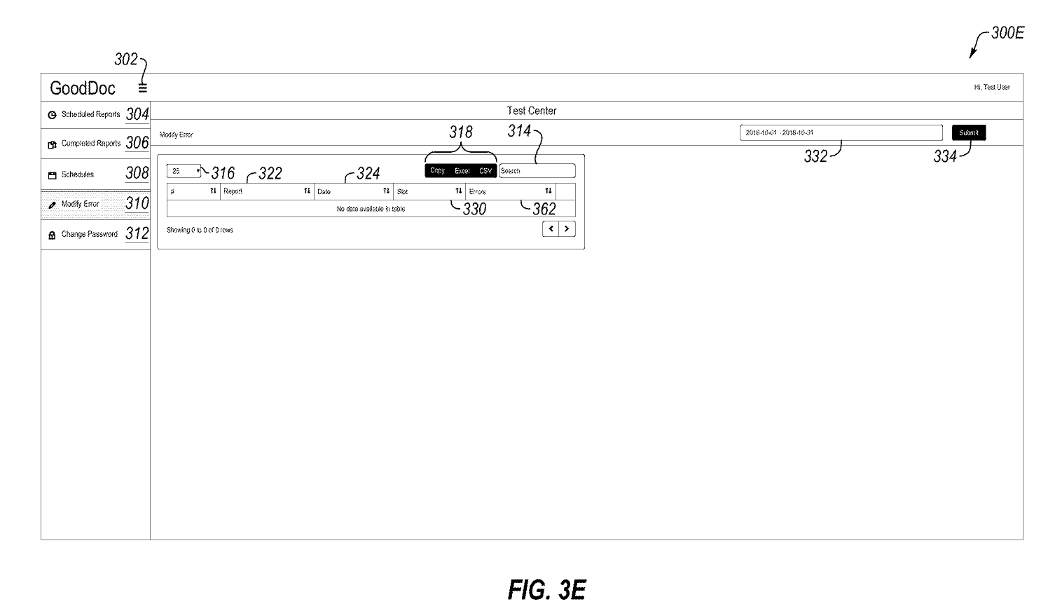

In some embodiments, the technician may have access to a local computing device 140, into which data may be entered as the technician reads values from the machines. In some embodiments, each of these local devices may be running a software application designed to receive data entries, analyze the entries, communicate message, determine trends, predict issues, and the like. An example user interface of the software application is described in more detail with reference to FIGS. 3A-3E, 4A-4G, and 5A-5F.

In some embodiments, as each data value is entered into the software, a communication or alert may be sent, in real-time to another party. In additional or alternative embodiments, communications or alerts may be delayed or held for a period of time based on a variety of reasons. Communications and alerts are described in more detail with reference to alert component 250.

In some embodiments, the technician may enter the time and the date associated with each data entry. In additional or alternative embodiments, the time and the date may be automatically obtained by a machine or a sensor (e.g., as received by or requested by the software application) associated with each data entry obtained by the data gathering component 240.

In some embodiments, alert component 250 may enable transmission of a communication regarding data values obtained and/or determined. In additional or alternative embodiments, alert component 250 may initiate an audio and/or visual alarm regarding data values obtained and/or determined.

In one example, data obtained at sensors 110 may fall within a pre-determined range to be considered acceptable and/or safe. In some embodiments, the lower- and upper-bounds of the ranges may be inclusive or exclusive. If a datum is obtained and determined to fall outside of an acceptable and/or safe range, a communication may be generated and transmitted, and/or an audio and/or visual alarm may activate. In additional or alternative embodiments, if a datum is determined to be outside of a pre-determined range or otherwise appears to be incorrect, a communication may be transmitted requesting confirmation of the datum. In additional or alternative embodiments, a datum may be acceptable, however, entry of the datum into the system may need a second confirmation; thus, a communication may be sent to two or more parties requesting confirmation that a value is acceptable.

In some embodiments, the pre-determined ranges and data may be dynamic and may vary from machine to machine, from day to day, from hour to hour. In additional or alternative embodiments, pre-determined ranges and data may be determined by government agencies, regulatory bodies, medical staff, the manufacturer of each machine, etc. In additional or alternative embodiments, the pre-determined ranges may be edited or changed by an administrator based on a variety of different reasons, including machine learning outputs, changes in laws and regulations, adjustments based on analysis with other clinics, standards, etc.

If the datum is determined to fall outside a pre-determined range, or if a datum is otherwise determined to trigger an alert, alert component 250 may generate a communication such as, but not limited to, a telephone call, a text message, an instant message, a pager alert, an e-mail, and the link. In some embodiments, the communication may be an audio and/or visual alert or an alarm, such as beeping, a siren, flashing lights, vibration, etc.

The communication and/or alarm may alert the receiver that attention is needed with respect to a specific patient, a specific datum, a specific device and/or machine, etc. In some embodiments, the communication may operate to automatically stop or start certain treatments.

In some embodiments, the communication may be sent to a nurse, a technician, a manager, a doctor, a third party, a government agency, etc. The communication may be sent in real-time (e.g., less than five seconds from obtaining the data, less than one minute, etc.) or may be sent if a follow-up action is not taken within a pre-determined time frame and/or if the datum is determined to still fall outside an acceptable range within a pre-determined time frame. In some embodiments, an alarm may activate (audio and/or visual), if the values are determined to be critical or within an emergency range.

In some embodiments, a communication may be sent and/or an alarm may be initiated if a data entry is not received by the logging component 230 within a pre-determined time frame (e.g., within 10 minutes of an expected data gathering time). If an initial communication and/or alert is not attended to within a pre-determined time frame, or if it is determined that the responsive action was not sufficient or proper, then the communications and/or alerts may escalate in intensity and/or may be sent to successively higher superiors.

In some embodiments, a communication may be sent and/or an alarm may be initiated if the expected number of entries is not met for a given time period, a given machine, etc. For example, if by 3:00 p.m., machine A should have four data entries in the software application, but only three entries have been made, a communication may be sent out and/or an alarm may be initiated to draw attention to the deficiency. Likewise, if four entries are expected by 3:00 p.m., but five entries have been provided to the application, then a communication may be sent and/or an alarm may be initiated to correct the error or confirm an intended number of entries.

Communications and alerts may be stored in memory, such as in a database, on any of the devices described with reference to FIG. 1, including, but not limited to, the local computing device 140, remote computing device 150, and/or server 130.

In some embodiments, analysis component 260 may enable data analysis in order to determine trends, predict failure of machine parts and/or components, compare data with other locations, provide analysis for government reporting, and the like. In some embodiments, data may be analyzed by comparing data entries with historical entries.

In one embodiment, the sensors 110 may be manufactured by and maintained by a third-parties. For example, a dialysis clinic may purchase or lease a water filter or a reverse osmosis device to be used in a water intake room, where water is initially received at the hospital from a municipal water source. In order to ensure the machines work property, parts may need to be replaced or fixed. Obtaining data from the machines may be analyzed to predict when parts may need replacement or service, and may aid in preventing failure of parts which would result in a loss of safety to the patient.

In one embodiment, obtained data may be analyzed to determine if there is an expected trend, or an unexpected trend, which would indicate failure. In some embodiments, obtained data may be analyzed to determine that although a filter has an example life of 10 months, the filter is likely to fail early or could still be safely used after the stated expiration date.

Currently, data gathering is done by hand, and as previously described, is subject to inaccuracies. In addition, multiple machines and devices may be monitored multiple times a day, a various locations, over a lengthy period of time (weeks, months, years), resulting in voluminous data that may not even be accurate and would be impossible to determine useful trends, predictions, or analysis using only pen-and-paper. Using the systems and methods described herein, the accuracy is improved, and trends may be determined at varying times and over varying time frames, such as over a day, over a week, over a month, over a year, etc. Furthermore, real-time analysis may be provided to a user at any time, as opposed to waiting for a lengthy attempt at predicting a trend using pen-and-paper.

In some embodiments, analysis and trending may be provided to a user as a visual representation, and easy-to-read displays such as, but not limited to, line graphs, bar charts, and pie diagrams. The user may also be presented with spreadsheets, and other user-friendly outputs.

In some embodiments, machine-learning algorithms may be employed to analyze data and determine trends, as well as predict potential failure of components, or to predict other data forecasting. Machine-learning may improve the accuracy of the data collection, as well as improve the analysis. Using machine-learning techniques, algorithms may be trained on some or all of the data from a single clinic, or data may be aggregated from multiple locations.

In some embodiments, data may be shared between clinics in a single location (e.g., a single hospital), across different hospitals or clinics in the same city or county, across clinics or hospitals in different states or different countries, and the like. In some embodiments, certain clinics may have been data gathering practices and analysis, alerts and communications, and satisfaction of regulations (whereas some clinics may be worse). Such data and considerations may be used to train machine-learning algorithms on data gathering and analysis at a different clinic or set of clinics in order to improve data gathering and processing of the data.

In some embodiments, data gathered by data gathering component 240 and/or analysis, trends, and/or predictions determined by analysis component 260 may be shared to a plurality of parties. In some cases, parties may be granted special access to gather, edit, read, manipulate, delete, save, share, etc., data with other parties. In some embodiments, a third party may be given temporary permission to receive communications and alerts from alert component 250. In this embodiment, the third party may be authorized to forward the communications and/or the data with the originally intended party for a set period of time. Once the time has elapsed, the third party may no longer have permission to access the data or receive communications.

The methods and systems described herein may be enabled for multiple different parties including, but not limited to, a technician, an administrator, a government agency, a corporate entity, etc. A software application may run on local computing device 140 and/or remote computing device 150 which may enable data entry, data manipulation, analysis, review, transmission of communications and alerts, receipt of communications and alerts, confirmation and validation, and other record keeping data. Depending on which party is given access to the software application, the user interface may be varied.

In some embodiments, the software application may be a webpage, whereas in other embodiments, the software application may be a downloadable application. Data may be entered while the application is connected to a network, thus facilitating communication, upload, download, storage, and retrieval of data in real-time. In other embodiments, data may be entered while the application is not connected to a network, and thus communication, storage, manipulation, analysis, etc. of data may be enabled when the application has network access.

Although example renditions are shown, the software need not be presented exactly as shown, and additional or fewer elements may be presented to a user. Although not specifically shown in FIGS. 3A-3E, 4A-4G, and 5A-5H, a user may first be presented with a secure log-in screen, where a user may enter a username and a password, or other secure identifying information to obtain access to the application. In some embodiments, the identifying information may be a username, an e-mail, a password, a personal identification number, a scannable barcode or other picture representation, and/or biometric information (e.g., fingerprint, voice print, retinal or iris scan, etc.).

FIGS. 3A-3E illustrate example interfaces related to cloud-based clinical logging, in accordance with at least one embodiment. More specifically, FIGS. 3A-3D illustrate example graphical user interfaces (GUIs) of the software application as accessed by a technician entering data values, among other options.

FIG. 3A shows a first display 300A. Although display 300A is shown first, display 300A is not necessarily the first screen a user may see upon signing in. In one embodiment, a toggle button 302 may be presented to the user which expands an example list of menu options 304, 306, 308, 310, and 312. In the embodiments shown in FIGS. 3A-3E, the expanded menu options are shown. A user may be presented with a plurality of menu options including, but not limited to, scheduled reports option 304, completed reports option 306, schedules option 308, modify errors option 310, and change password option 312.

FIG. 3A illustrates the scheduled reports option 304. In one embodiment, a user may interact with the display 300A to enter, sort, and/or manipulate a plurality of data. In particular, scheduled reports may indicate to a user a list of open reports for which data should be entered. The list may include, but is not limited to, both late and missed reports. For example, an entry number 322, a name of a report 324, the date of the entry 326, the time of the entry 328, a status 330, and additional options 332.

Entry number 322 may be a numerical value indicative of a chronological order of entries entered by the technician. Status 330 may represent the current status of the log, such as completed, not complete, or in progress. Other status options may be contemplated and are not limited to the three provided.

In addition, the user may sort the data in an ascending or descending order using any of the data fields as the primary sorting factor. The user may further determine how many data entries to see at one time by interacting with button 316. For example, in one embodiment, it may be beneficial for the user to see the last 25 entries sorted by time, whereas in another embodiment, it may be beneficial for the user to see the last 100 entries sorted by status. In addition, the user may use the search field 314 to search for a particular entry or data field.

In some embodiments, the user may wish to obtain a printed (digital or hardcopy) report of selected data. Thus, the user may select an output format option 318, for example, copying the data, generating a spreadsheet, or generating a table. Although three options are shown, any number of output options may be presented to the user.

FIG. 3B illustrates the completed reports option 306. In one embodiment, a user may interact with the display 300B to enter, sort, and/or manipulate a plurality of data, as previously described with reference to FIG. 3A. In particular, completed reports 306 may be viewed individually, as well as collectively. For example, the user may request to see completed reports for a requested date range 332 (entered by actuating an example “submit” button 338). The completed reports may include, but are not limited to, an entry number 322, a name of a report 324, the date of the report 326, and a time slot 330 over which the log has been obtained.

In some embodiments, the user may enter a date range 332 (e.g., by entering a date range and actuating a “submit” button 334) to populate the table of screen 300B with completed reports for the entered date range.

In some embodiments, entry number 322 and a name of a report 324 correspond to the same columns of scheduled reports 304, respectively, such that there is continuity between what the user sees from one screen to the next. In other words, an entry number 322 and/or name of a report 324 from the scheduled reports 304 menu may later correspond to an entry number 322 and/or name of a report 324 in the completed reports option 306, after the technician completes data entry for the scheduled report.

FIG. 3C illustrates a first version of the schedules option 308. In one embodiment, a user may interact with the display 300A to enter, sort, and/or manipulate a plurality of data (as described previously with reference to FIG. 3A). In some embodiments, if a user (e.g., a manager and/or administrator) has not yet scheduled a report, schedules option 308 enables a feature to schedule a report. In some embodiments, reports may be scheduled with limited capacity.

In some embodiments, the user may select a log from the drop down menu 336. The log may include, for example, logs related to, but not limited to, a bicarbonate concentration system log, a total chlorine log, a water system log, etc. In addition, the user may enter a start time 338, an end time 340, remarks regarding an entry or a scheduling decision into a text box 342, an a PIN 344. In some embodiments, the PIN 344 may be a secure identifying signature unique to a user entering data and may be used to identify who has scheduled the report, entered data, manipulated data, etc.

The start time 338 may be the time at which a specific test begins and the end time 342 may be the time at which the specific test ends. In some embodiments, the start time and the end time may be entered in a 24 hour format.

In one embodiment, the user may be presented with multiple display options for scheduling: a calendar option 346 and a table option 348. If the user selects the calendar option 346, the user may be presented with a calendar 350 as shown in FIG. 3C. Tests may be displayed within the calendar for the user to select based on the data. If the user selects the table option 348, the user may be presented with a table as shown in FIG. 3D.

FIG. 3D illustrates a second version of the schedules option 308. In this embodiment, the user has selected the table option 348 and has been presented with a table similar to tables shown with respect to FIGS. 3A and 3B. For example, display 300D may present the user with an entry number 322, a log number 352, a start date 356, an end date 358, a time 358, and a frequency 360. Entry number 322 may represent the entry made by the technician in a chronological order, regardless of any other data; whereas the log 352 may represent a specific identifier for a specific log. The log 352 value may be pre-determined by an administrator, a manufacturer, a third party, etc. In FIG. 3D, the start date 354 and the end date 356 may represent the date range for a specific test (as selected in dropdown menu 336), whereas the time 358 may represent the time the log was taken The frequency may represent how many times over a time period the data entries should be entered for a specific log.

Dropdown menu 368 may expand to present the user with the available logs from which the user may make a selection. For example, the available logs 386A may include, but are not limited to, a dialysis machine disinfection log, citric acid mixing, temperature, specific gravity, bicarbonate mixing, total chlorine, water room temperature, back flow, incoming water pressure, incoming water temperature, sediment tank delta pressure, softener delta pressure, salt level in a brine tank, pre-softener hardness, post-softener hardness, primary carbon tank delta pressure, secondary carbon tank delta pressure, empty bed contact time for carbon tanks, permeate flow rate, concentrate flow rate, recycle flow rate, primary reverse osmosis membrane pressure, pyrogen delta pressure, ultra-filter delta pressure, ultraviolet light testing, flow velocity, recirculation pump, glucose meter, refrigerator temperature, and the like.

FIG. 3E illustrates a modify error option 310. In one embodiment, a user may interact with the display 300E to enter, view, sort, and/or manipulate a plurality of data (as described previously with reference to FIG. 3A). The user may enter a date range 332 in order to obtain a specific set of reports from which to make a selection. In addition to an entry number 322, a name of a report 324, a date 326, and a slot 330, an error 362 column may also be shown. In one embodiment, the user may select an entry in order to correct any potential errors displayed in column 362. For example, the technician may have entered an incorrect value into the software application at some point, and when a communication or alert was sent to second party for verification, the second party indicated a likely error. Although a specific screen is not shown for the change password 312 menu option, the software application may present the user with the option to change a password, a user name, or other account or profile information. Other displays may be presented to the user, although not specifically shown, which may enable the user to communicate with other parties, seek help, etc.

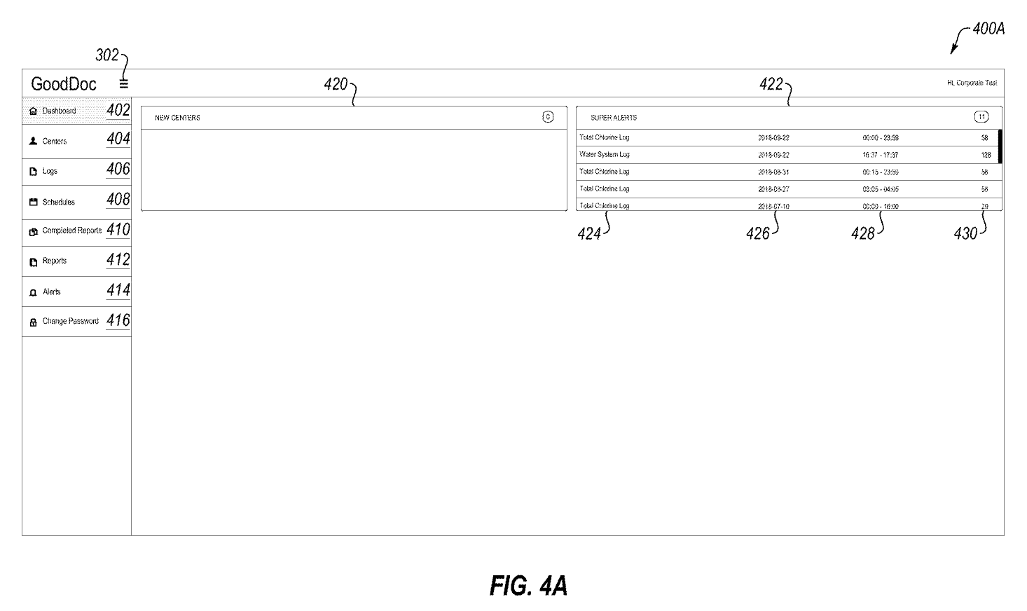

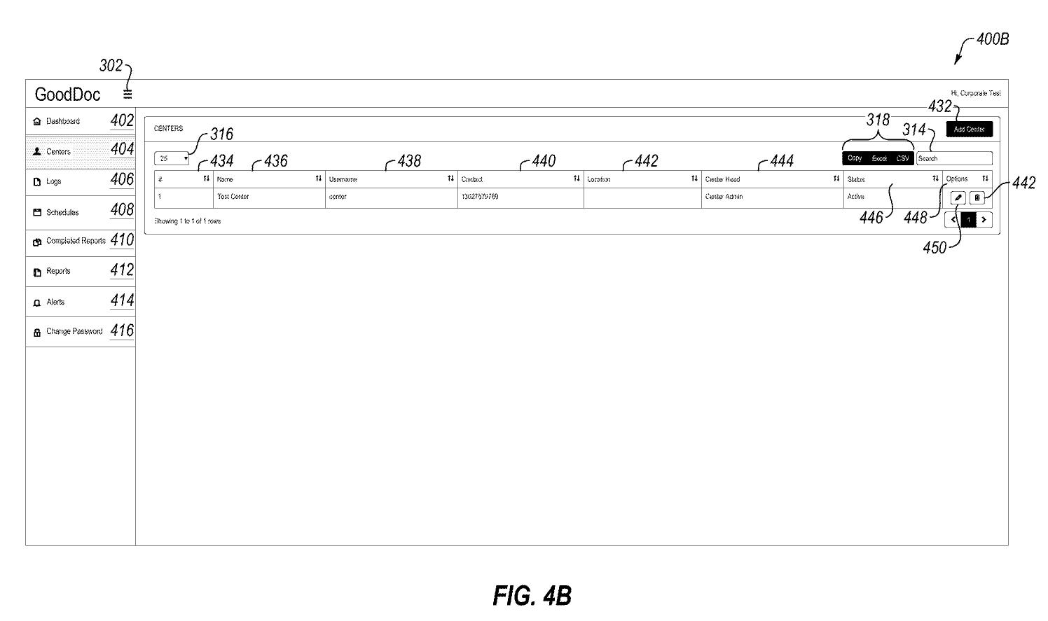

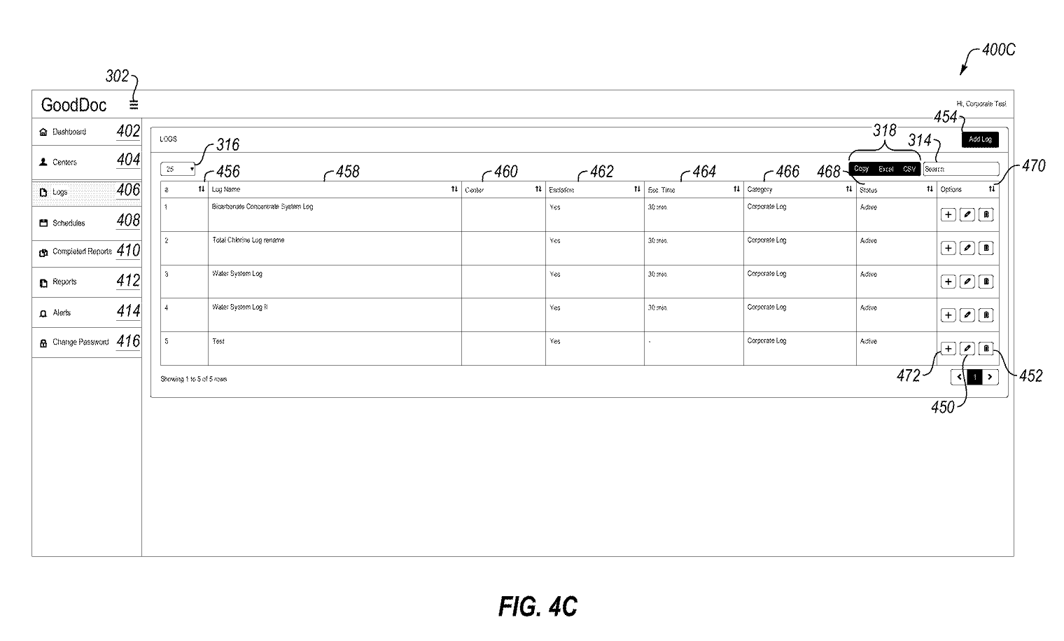

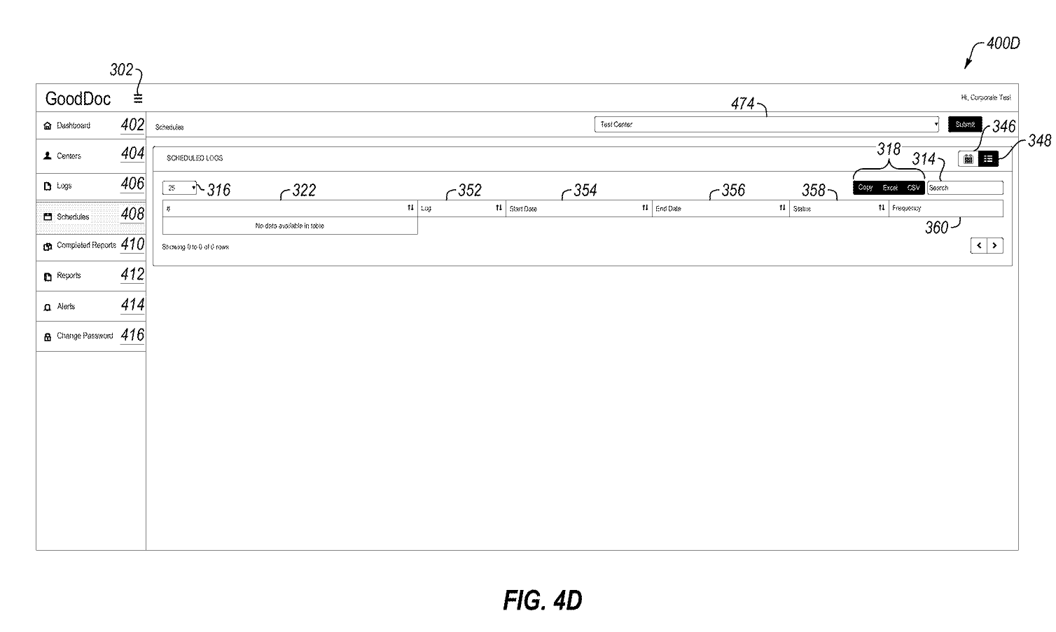

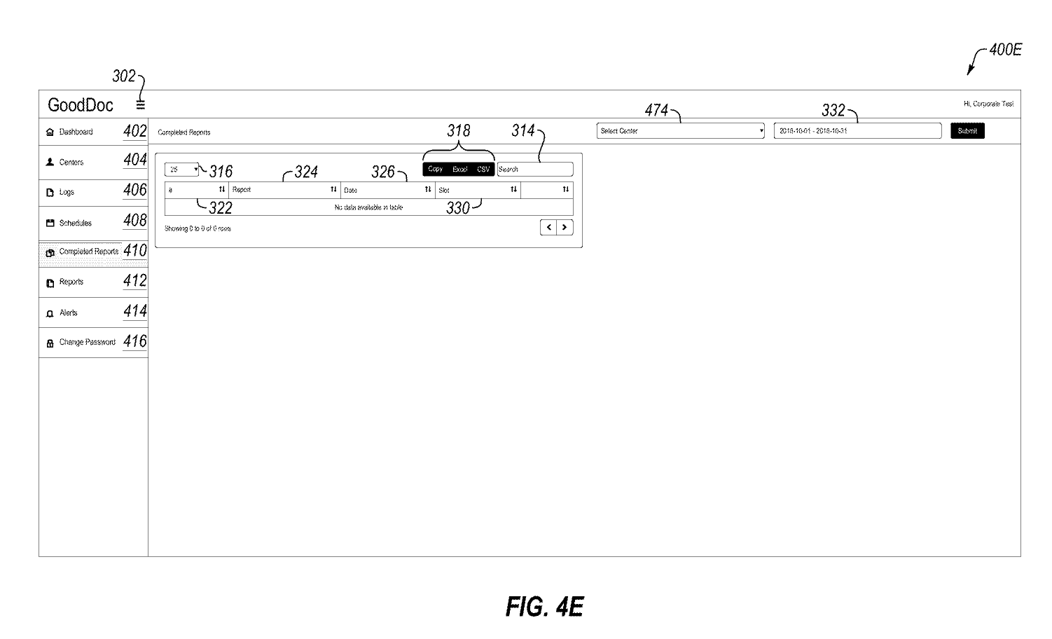

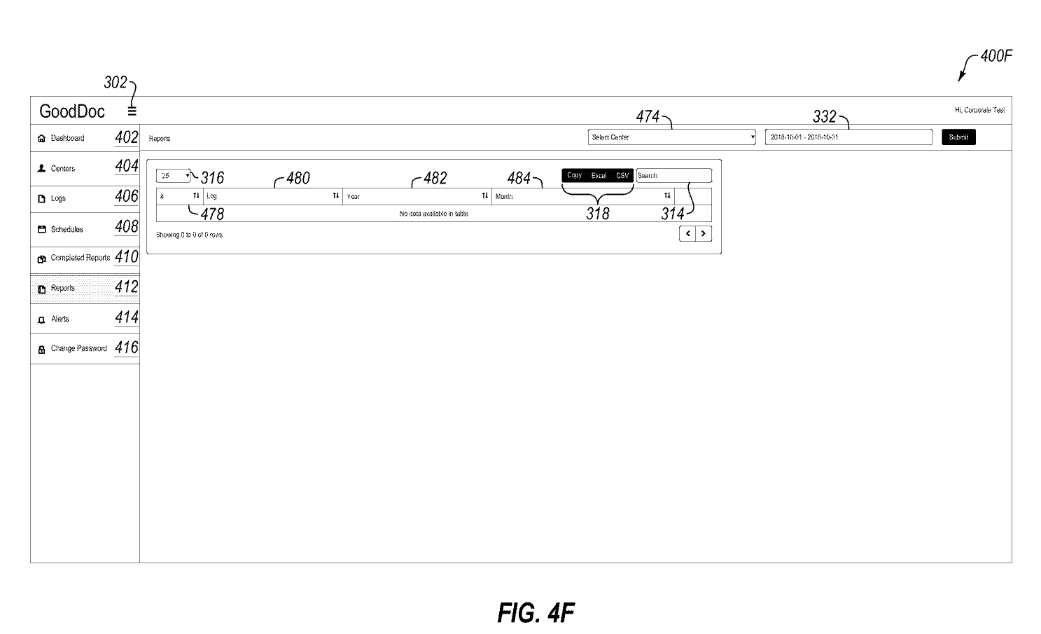

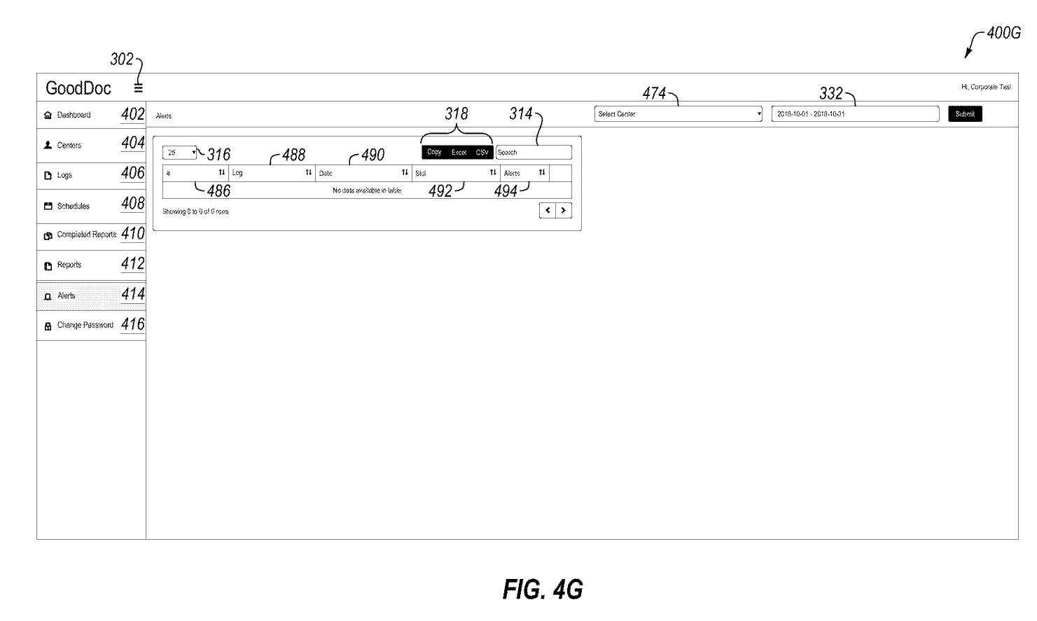

FIGS. 4A-4G illustrate example interfaces related to cloud-based clinical logging, in accordance with at least one embodiment. More specifically, FIGS. 4A-4G illustrate example graphical user interfaces (GUIs) of the software application as accessed by a corporate manager or government agency, etc., in order to review entries provided by a technician at a clinic.

FIG. 4A shows a first display 400A. Although display 400A is shown first, display 400A is not necessarily the first screen a user may see upon signing in. In one embodiment, a toggle button 302 may be presented to the user which expands an example list of screen options 402, 404, 406, 408, 410, 412, 414, and 416. In the embodiments shown in FIGS. 4A-4G, the expanded menu options are shown. A user may be presented with a plurality of menu options including, but not limited to, a dashboard option 402, clinics option 404, logs option 406, schedules option 408, completed reports option 410, reports option 412, alerts option 414, and a change password option 416.

Display 400A presents a user with two example windows: a new clinics 420 window and an alerts 422 window. In some embodiments, the dashboard option 402 presents the user with an overview and/or summary of clinics as well as alerts of which to take note.

In some embodiments, as new clinics are added, they may appear in the new clinics 420 window. New clinics may benefit from additional scrutiny, and thus having a quick summary of new clinics may provide an administrator with an easy view.

Alerts 422 window may present the administrator with a list of alerts or communications indicating values that may require attention. A list of logs 424 may be presented, along with a corresponding date 426 and time range 428 of each alert. Column 430 may illustrate the number of alerts within the displayed date 426 and time range 428. For example, alerts 422 window shows an example five alerts: four total chlorine log alerts and a water system log alerts. Further, the first entry for total chlorine log shows 58 example alerts on September 22, 2018 between the hours of midnight and 11:59 p.m.

FIG. 4B may illustrate a list of clinics that are being monitored or for which alerts and communications are being logged. Display 400B may present the user with an option 432 to add a clinic to the logging list. In some embodiments, selecting the option 432 may present the user with another window to add identifying information about a clinic, including a name, address, contact name, contact information, status, and additional options.

Display 400B shows an example clinic having an entry number 434, a name 436, a username 438, a contact 440, a location 442, a clinic head 44, a status 446, and options 448. Options 448 may include the option to edit 450 and to delete 452 the selected clinic. As described previously, the entry number 434 may simply be a number that corresponds chronologically with each clinic that is added to the logging. The name 436 may be the name of the clinic, whereas a username 438 may be a username selected by someone working at the clinic or may be a default username to associate the clinic with the software application. Contact 440 may be a contact phone number or email (or other contact information) for an administrator to contact an administrator or a clinic head 444 at the clinic. Location 442 may be the address or other location identifier of the clinic. In some embodiments, clinic information may be stored even if values are not being logged currently; thus, a status 446 may be indicated, where example options may include active or not active.

FIG. 4C may illustrate a list of logs for all of the clinics or some of the clinics. Display 400C may present the user with a table that includes, but is not limited to, an entry number 456, a log name 458, a clinic 460, escalation 462, an escalation time 464, a category 466, a status 468, and options 470. In some embodiments, such as from the perspective of a corporate user, if a value entered does not satisfy the pre-determined range, the “escalation” option is activated if a technician does not respond within a predetermined time frame or in a pre-determined way. Once the escalation 462 option is activated, a director or other supervisor may be notified. Other users notified may include, but are not limited to, a director of operations, a head of technical services, and/or other management personnel.

Options 470 may include the option 472 to add additional information to the logs, if needed, the option 450 to edit an entry, and the option 452 to delete an entry. Furthermore, the user may select the option 454 to add a log that is not already present.

As before, the entry number 456 may be automatically assigned based on the chronological input of data. The log name 458 may correspond to the logs described with reference to FIGS. 3C and 3D. Example logs may include, but are not limited to, a bicarbonate concentrate system log, a total chlorine log, a water system log, etc. Each entry may be associated with a specific clinic 460. In some embodiments, the escalation time 464 may be modifiable and/or determined by a head office and/or administrator. In addition, a category 466 may represent whether a log was created by the corporate office administrator or by an individual clinic. A log status 468 may indicate of the log is actively being updated, or in an additional or alternative embodiment, whether the log is historic and/or has been suspended.

FIG. 4D illustrates a list of scheduled logs. FIG. 4D may correspond to the scheduled logs described with reference to FIGS. 3C and 3D. For example, FIG. 4D illustrates a first version of a scheduled log option. For example, the user may select a clinic from the drop down menu 474. In one embodiment, the user may be presented with multiple display options for scheduling: a calendar option 346 and a table option 348. If the user selects the calendar option 346, the user may be presented with a calendar similar to the calendar shown in FIG. 3C. If the user selects the table option 348, the user may be presented with a table as shown in FIG. 4D.

For example, display 400D may present the user with an entry number 322, a log number 352, a start date 356, an end date 358, a time 358, and a frequency 360. Entry number 322 may represent the entry made by the technician in a chronological order, regardless of any other data; whereas the log 352 may represent a specific identifier for a specific log. The log 352 value may be pre-determined by an administrator, a manufacturer, a third party, etc. In FIG. 4D, the start date 354 and the end date 356 may represent the date range for a specific test (as selected in dropdown menu 336), whereas the time 358 may represent the time of the entry. The frequency may represent how many times over a time period the data entries should be entered for a specific log.

FIG. 4E illustrates a list of completed reports. FIG. 4E may correspond to the completed reports described with reference to FIG. 3B. The administrator may select a specific clinic from the dropdown menu 474, which may display the completed reports on display 400E that are shown to the user on display 300B (if 300B shows the completed reports for the selected clinic). The administrator may also select a date range 332. For example, the user may request to see completed reports for a requested date range 332 (entered by actuating an example “submit” button 338). The completed reports displayed in display 400E may include, but are not limited to, an entry number 322, a report number 324, the date 326, and a slot 330, which may be as described with reference to FIG. 3B.

FIG. 4F illustrates a list of reports. In previously described embodiments, “completed reports” may include individual report entries; in contrast, display 400F may show a compilation of previously completed reports, which may be available in a single downloadable file. As described previously, the user may select a specific clinic from the dropdown menu 474. Additionally, the user may select a date range 332 in order to display the list of reports for the selected clinic for the date range. Display 400F may illustrate an entry number 478, a log number 480, a year 482, and a month 484 associated with each of the report entries in the list.

FIG. 4G illustrates a list of alerts. As previously described, the user may select a specific clinic from the dropdown menu 474, as well as a selected date range 332. Based on the selection, the user may be presented with a list of alerts. Display 400G may provide the user with an entry number 486, a log number 488, a date 490 when the alert occurred, a slot 492 which represents the start and end time of the entry, and a number of alerts 494 for the corresponding entry number 486. In one example, the alert 494 may provide the user with information that a communication or alert was initiated based on a recorded value falling outside of an acceptable range. In some embodiments, the display 400G may only provide an indication there was an alert; however, in an additional or alternative embodiment, the display 400G may provide specific information about the alert, including what the values were, to whom a communication was sent, and if there was a response, follow-up, or correction made.

Although a specific display is not shown for the change password option 416, the software application may present the user with the option to change a password, a user name, and/or other profile information. Other displays may be presented to the user, although not specifically shown, which may enable the user to communicate with other parties, seek help, etc.

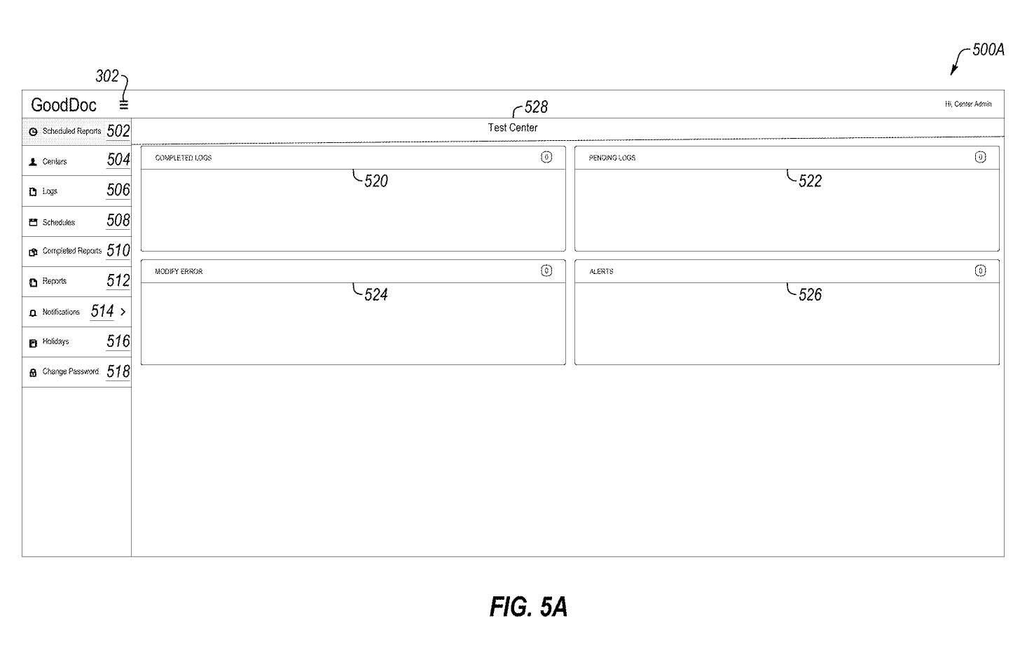

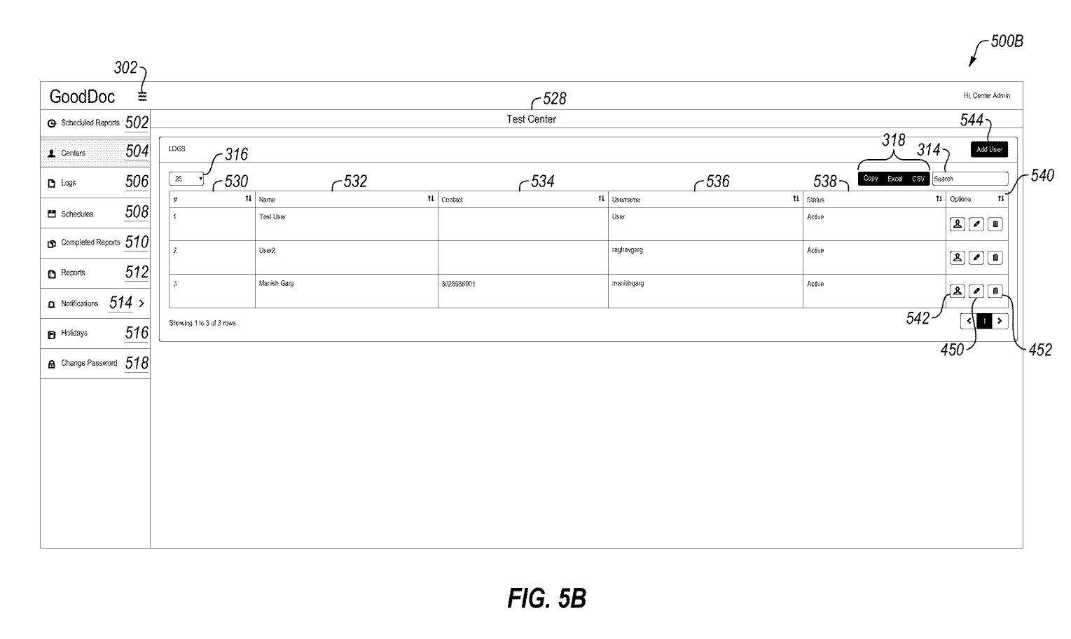

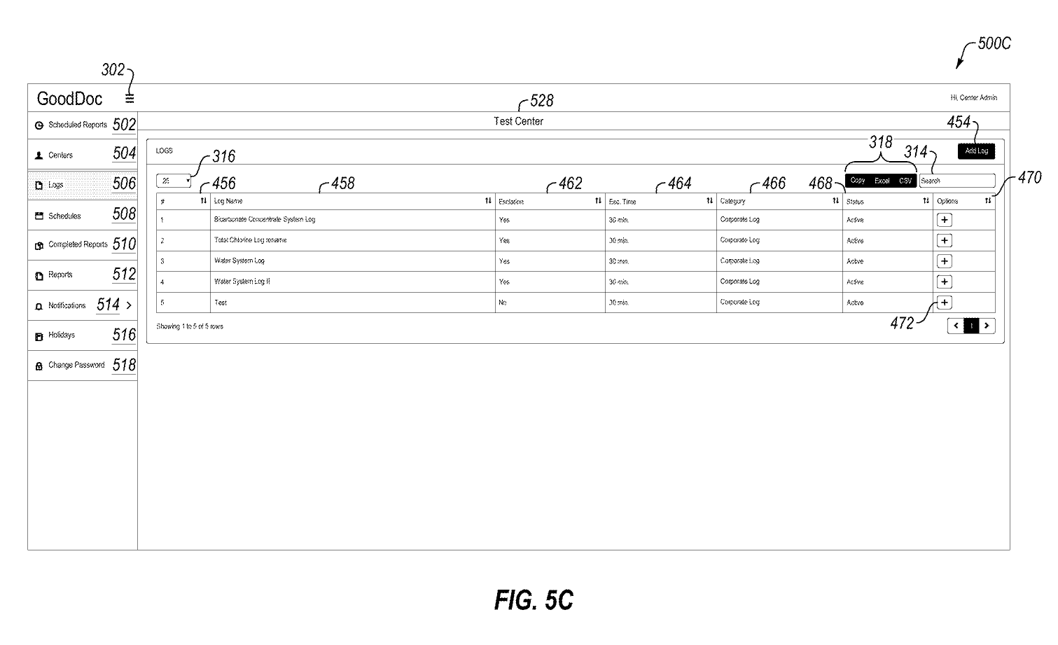

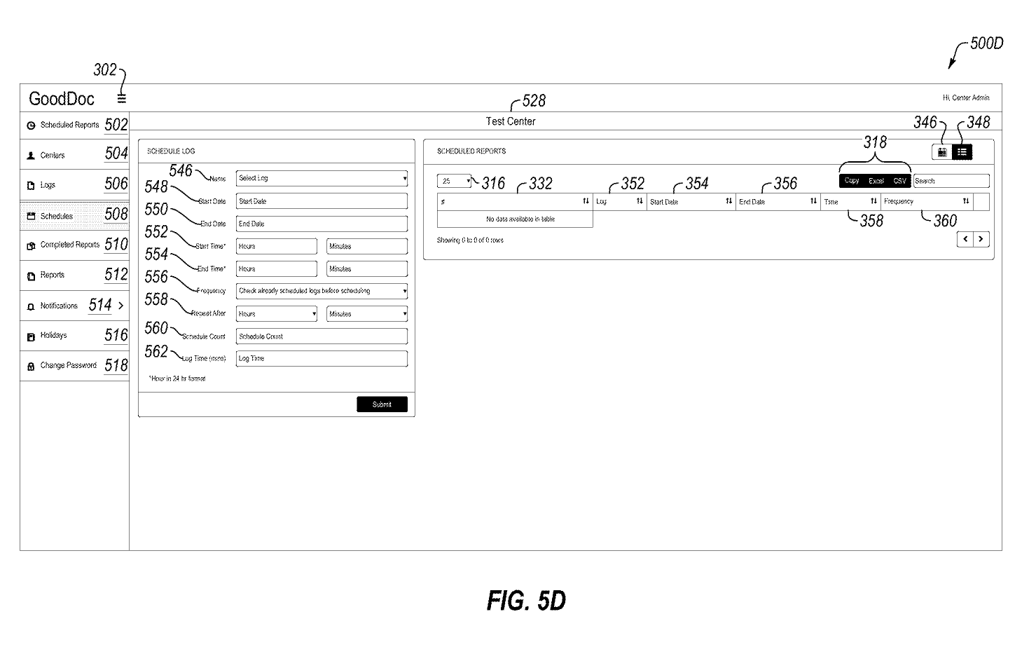

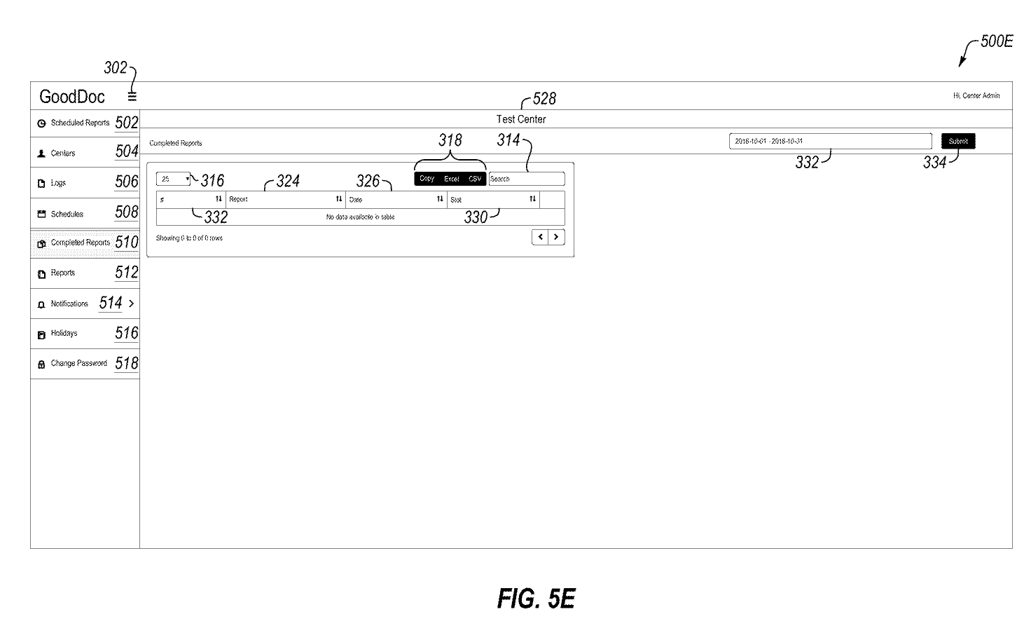

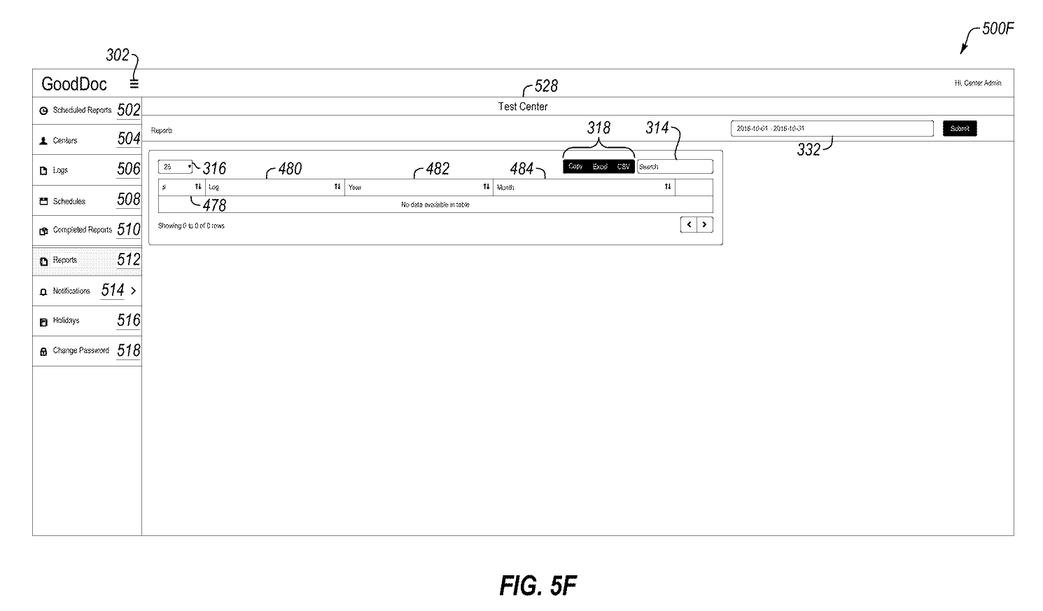

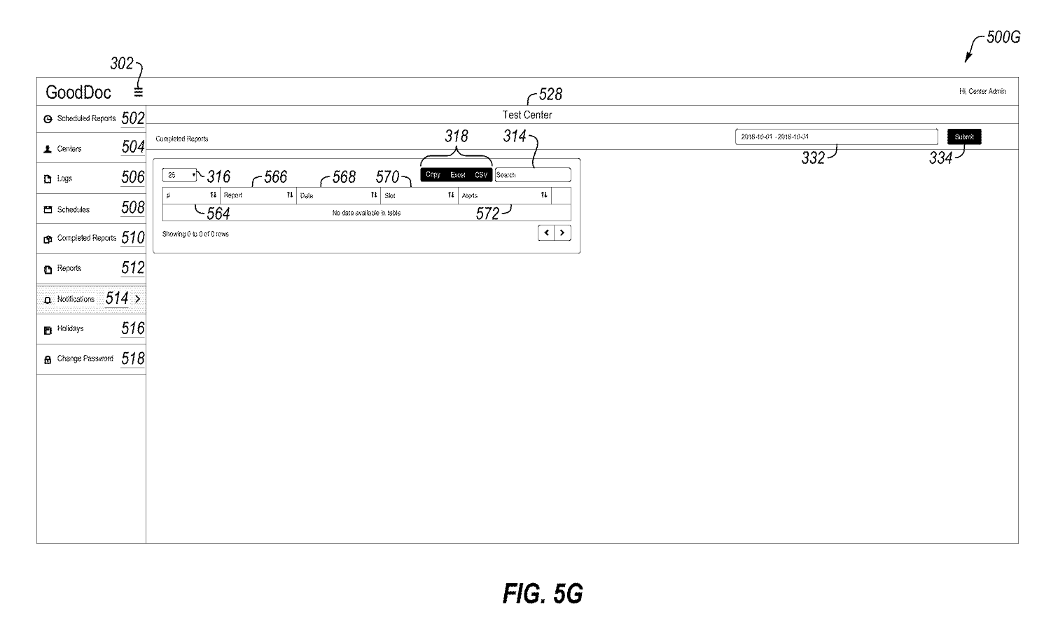

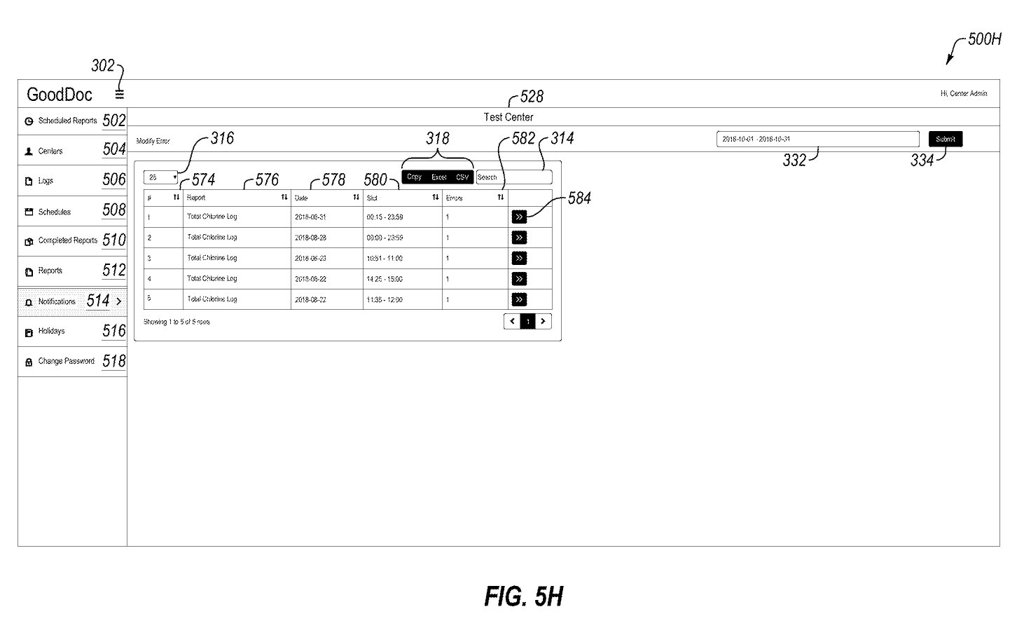

FIGS. 5A-5H illustrate example interfaces related to cloud-based clinical logging, in accordance with at least one embodiment. More specifically, FIGS. 5A-5H illustrate example graphical user interfaces (GUIs) of the software application as accessed by an administrator at a specific clinic in order to review entries provided by a technician at the clinic.

FIG. 5A shows a first display 500A. Although display 500A is shown first, display 500A is not necessarily the first screen a user may see upon signing in. In one embodiment, a toggle button 302 may be presented to the user which expands an example list of screen options 502, 504, 506, 508, 510, 512, 514, 516, and 518 for a specific clinic 528 with which an administrator is associated. In the embodiments shown in FIGS. 5A-5H, the expanded menu options are shown. A user may be presented with a plurality of menu options including, but not limited to, a dashboard option 502, a users option 504, a logs option 506, a schedules option 508, a completed reports option 510, a reports option 512, a notifications option 514, a holidays option 516, and a change password option 518.

Display 500A may illustrate the dashboard option 502, and in one example may present a user with four example windows: a completed logs window 520, a pending logs window 522, a modify error window 524, and an alerts window 526. As data is entered by a technician at the clinic 528 into the software application, or as data is automatically obtained by the software application, the windows 520, 522, 524, and 526 may be populated with entries from which the administrator may make selections or garner an overall summary of the data. In some embodiments, the administrator may be able to select any of the entries displayed in the windows 520, 522, 524, and 526, or otherwise sort the entries.

FIG. 5B illustrates an example list of users associated with the clinic 528. Display 500B may provide the administrator with a list of users that have entered data into the software application at clinic 528, or have otherwise interacted with the data in some way (e.g., edited entries, deleted entries, added entries, validated entries, etc.).

Entry number 530 may be similar to entry numbers previous described, and may be automatically assigned based on adding a user. In some embodiments, a user may be added by selecting the add user option 544. Selecting the add user option 544 may present the administrator with a window to enter information about a user to be added such as, but not limited to, a name, username, contact information, permissions, access, a status, etc.

Display 500B may present the administrator with the user's name 532, contact information 534, a username 536, a status 538 (e.g., active, inactive, a level of access, etc.), and options 540. Options 540 may include a profile option 542 to view more information about a specific user, an edit option 450, and a delete option 452.

FIG. 5C illustrates an example list of logs associated with the clinic 528. Display 500C may be similar to, or related to, the logs described with reference to 4C. Display 500C may present the user with a table that includes, but is not limited to, an entry number 456, a log name 458, a clinic 460, escalation 462, an escalation time 464, a category 466 which may represent whether a log was created by the corporate office administrator or by an individual clinic, a status 468, and options 470. Options 470 may include the option 472 to add more information to each log, the option 450 to edit an entry, and the option 452 to delete an entry. Furthermore, the user may select the option 454 to add a log that is not already present.

As before, the entry number 456 may be automatically assigned based on the chronological input of data. The log name 458 may correspond to the logs described with reference to FIGS. 3C, 3D, and 4D. Example logs may include, but are not limited to, a bicarbonate concentrate system log, a total chlorine log, a water system log, etc. Each entry may be associated with a specific clinic 460. Escalation 462 and escalation time 464 are similar to the escalation and escalation times described previously. In addition, a category 466 may represent whether a log was created by the corporate office administrator or by an individual clinic. A log status 468 may indicate of the log is actively being updated, or in an additional or alternative embodiment, whether the log is historic and/or has been suspended.

FIG. 5D illustrates an example schedule log associated with the clinic 528. Display 500D may be similar to, or related to, the schedule log of FIGS. 3C, 3D, and 4D. In one embodiment, the user may be presented with multiple display options for scheduling: a calendar option 346 and a table option 348. If the user selects the calendar option 346, the user may be presented with a calendar similar to the calendar shown in FIG. 3C. If the user selects the table option 348, the user may be presented with a table as shown in FIG. 4D and by display 500D.

The administrator may schedule a log by selecting a pre-determined log from the dropdown menu 546, a start date 548, an end date 550, a start time 552, an end time 554, a frequency 556, a repeat option 558, a schedule count 560, and a log duration 562. Thus, when scheduling an intended log, the administrator may specify the date range over which the data should be logged (i.e., start date 548 and end data 550), a specific time range over which the data should be logged (i.e., start time 552 and end time 554), and how often within the date and time ranges the data should be obtained (i.e., frequency 556). In some embodiments, the administrator may select an option 558 to repeat the logging after a specified period of time, such as every hour, or every 90 minutes, etc. In some embodiments, the administrator may specify a schedule count 560, which may represent the number of logs to be scheduled. In some embodiments, the administrator may specify a log time 562, which may determine the duration for which the log is available to be completed.

Multiple log features enable multiple logs in a single day to be linked with each other; for example, a chlorine log may have reports four times a day, with each entry being within four hours of the previous entry—by enabling multiple scheduling, each subsequent log will be scheduled in a manner that follows predetermined scheduling guidelines.

In addition, display 500D may present the user with an entry number 322, a log number 352, a start date 356, an end date 358, a time 358, and a frequency 360. Entry number 322 may represent the entry made by the technician in a chronological order, regardless of any other data; whereas the log 352 may represent a specific identifier for a specific log. The log 352 value may be pre-determined by an administrator, a manufacturer, a third party, etc. In FIG. 5D, the start date 354 and the end date 356 may represent the date range for a specific test (as selected in dropdown menu 336), whereas the time 358 may represent the time the test occurred. The frequency may represent how many times over a time period the data entries should be entered for a specific log.

FIG. 5E illustrates a list of completed reports for clinic 528. Display 500E may be similar to, or related to, the completed reports described with reference to FIGS. 3B and 4E. The administrator may select a date range 332; for example, the user may request to see completed reports for a requested date range 332 (entered by actuating an example “submit” button 338). The completed reports displayed in display 500E may include, but are not limited to, an entry number 322, a name of a report 324, the date 326, and a slot 330, which may be as described with reference to FIGS. 3B and 4E.

FIG. 5F illustrates a list of reports for clinic 528. Display 500F may be similar to, or related to, the reports described with reference to FIG. 4F. As described previously, the user may select a specific clinic from the dropdown menu 474. Additionally, the user may select a date range 332 in order to display the list of reports for the selected clinic for the date range. Display 400F may illustrate an entry number 478, a log number 480, a year 482, and a month 484.

FIGS. 5G and 5H may illustrate different notifications and alerts for clinic 428. Displays 500G and 500H may be similar to, or related to, the alerts described with reference to FIG. 4G. When looking at different notifications and alerts, the administrator may select from a number of notification options such as alerts, missed logs, issues, and an opportunity to modify errors. FIG. 5G shows an example of a list of alerts. The administrator may select a date range 332 of alerts to display for clinic 528. Display 500G may present an entry number 564, a log number 566, a date 568, a slot 570, and the corresponding alert 572. In one example, the alert 572 may provide the user with information that a communication or alert was initiated based on a recorded value falling outside of an acceptable range. In some embodiments, the display 500G may only provide an indication there was an alert; however, in an additional or alternative embodiment, the display 500G may provide specific information about the alert, including what the values were, to whom a communication was sent, and if there was a response, follow-up, or correction made.

FIG. 5H may illustrate an example list of errors that may need attention. Display 500H may be similar to, or related to, FIG. 3E. The user may enter a date range 332 in order to obtain a specific set of logs from which to make a selection. In addition to an entry number 574, a log number 576, a date 578, a slot 580, and a count 552. In one embodiment, the administrator may select an entry in order to correct any potential errors (e.g., such as by selection option 584). For example, the technician may have entered an incorrect value into the software application at some point, and when a communication or alert was sent to second party for verification, the second party indicated a likely error.

Additional displays and options may be presented with respect to various versions of the software application presented in FIGS. 3A-3E, 4A-4G, and 5A-5H, including adding holidays, viewing analytics and trends, ways to aggregate and communicate information, request additional information, etc. Thus, the options presented in FIGS. 3A-3E, 4A-4G, and 5A-5H should be considered mere examples, and not be considered exclusive.

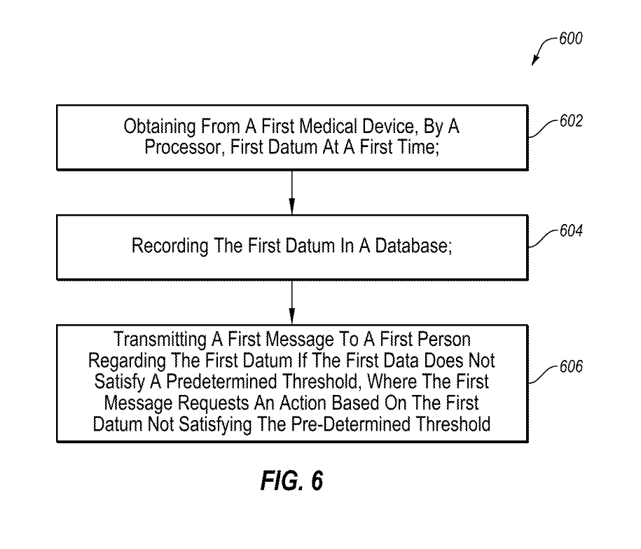

FIG. 6 illustrates a flowchart of an example method related to cloud-based clinical logging, in accordance with at least one embodiment. The method 600 may be performed, in some embodiments, by a device or system, such as by the sensors 110, local computing device 140, remote computing device 150, and/or the server 130. In these and other embodiments, the method 400 may be performed by one or more processors based on one or more computer-readable instructions stored on one or more non-transitory computer-readable media. The method 600 will now be described in connection with FIGS. 1, 2, 3A-3E, 4A-4G, and 5A-5H.

At action 602, the method may include obtaining from a first medical device, by a processor, a first datum at a first time. For example, a sensor on a first medical device may automatically obtain values associated with the medical device. In one embodiment, the medical device may be one of a plurality of water purification devices, and the sensor may determine, for example, an initial water temperature of water entering into the water purification device. The first value may be obtained at a requested sample time, which may be mandated by an agency, such as the United States Department of Health.

At action 604, the method may include recording the first datum in a database. For example, the first datum obtained may be stored in a database or a log so that the obtained data can be analyzed and other determinations can be made. The database or log may be part of a software application or program running on a computing device, such as a smartphone, laptop, or desktop computer.

At action 606, the method may include transmitting a first message to a first person regarding the first datum if the first data does not satisfy a predetermined threshold, where the first message requests an action based on the first datum not satisfying the pre-determined threshold. For example a first message may include a text message, a page, an e-mail, a telephone call, a voice message, an instant message, or some combination thereof. The message may be sent to a technician, an administrator, a supervisor, a doctor, a nurse, a corporate party, a third-party (e.g., a manufacturer of the medical device), and the like. The pre-determined threshold, for example, may be a range of values determined to be medically acceptable, and may be determined by an agency (e.g., Department of Health), a doctor, a nurse, a technician, an administrator, etc. If the datum is determined to fall outside of the medically acceptable (or safe) pre-determined range, or if the range, establishes a range that does not require a follow-up action, then the message may be sent to a party to indicate to the party a request to do an action. The action may be to confirm the accuracy of the value, make an adjustment to the machine, inform a second person, stop a treatment for a patient, or some other action.

In some embodiments, the methods and systems described herein may use machine learning techniques to predict trends. In addition, the methods and systems described herein may combine a series of steps that are unconventional in order to provide safe and accurate medical treatment to patients. By obtaining, analyzing, and following-up on obtained data, the computer systems associated with medical devices, as well as patient treatment, may be improved. For example, the accuracy at which certain elements of treatment can be adjusted may be improved by providing more accurate data both in the determination of values, and the time and frequency in which values are obtained. In some cases, the methods and systems described herein may improve accuracy in such a way as to facilitate automation of treatment and/or adjustment of medical devices.

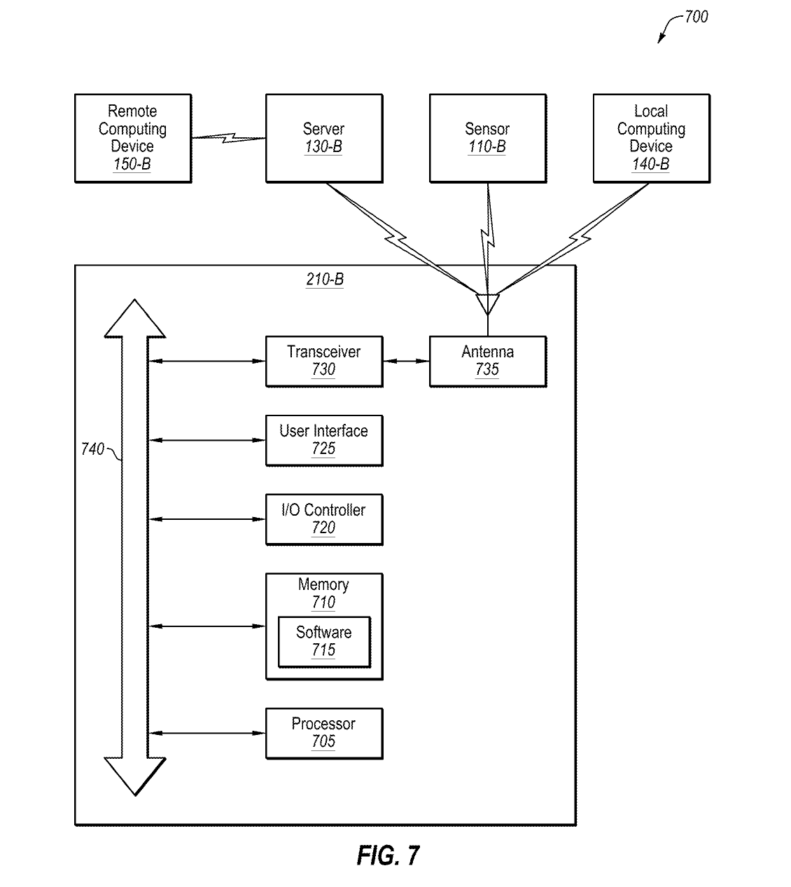

FIG. 7 illustrates an example computer system for cloud-based clinical logging, in accordance with at least one embodiment. In some embodiments, the computer system 700 may be part of any of the systems or devices described in this disclosure. For example, the computer system 700 may be part of any of sensors 110, local computing device 140, remote computing device 150, server 130, and apparatus 210 of FIGS. 1 and 2. Apparatus 210-b may also be an example of one or more aspects of apparatus 210 described with reference to FIG. 2.

Apparatus 210-b may include components for bi-directional audio, visual, and data communications including components for transmitting communications and data and components for receiving communications and data. For example, apparatus 210-b may communicate bi-directionally with one or more of local computing device 140-b, one or more sensors 110-b, remote computing device 150-b, and/or server 130-b, which may be an example of the components described with reference to FIG. 1. This bi-directional communication may be direct (e.g., apparatus 210-b communicating directly with remote computing device 150-b) or indirect (e.g., apparatus 210-b communicating indirectly with remote computing device 150-b through server 130-b).

Apparatus 210-b may also include a processor 705, and memory 710 (including software/firmware code 715), an input/output controller 720, a user interface 725, a transceiver 730, and one or more antennas 735 each of which may communicate—directly or indirectly—with one another (e.g., via one or more buses 540). The transceiver 730 may communicate bi-directionally—via the one or more antennas 735, wired links, and/or wireless links (e.g., communication links 160 described with reference to FIG. 1)—with one or more networks or remote devices as described above. For example, the transceiver 730 may communicate bi-directionally with one or more of sensors 110-b, local computing device 140-b, remote computing device 150-b, and/or server 130-b. The transceiver 730 may include a modem to modulate the packets and provide the modulated packets to the one or more antennas 735 for transmission, and to demodulate packets received from the one or more antennas 735. While the apparatus 210-b may include a single antenna 735, the apparatus may also have multiple antennas 735 capable of concurrently transmitting or receiving multiple wired and/or wireless transmissions. In some embodiments, one element of apparatus 210-b (e.g., one or more antennas 735, transceiver 730, etc.) may provide a direct connection to the server 130-b via a direct network link to the Internet via a POP (point of presence). In some embodiments, one element of apparatus 210-b (e.g., one or more antennas 735, transceiver 730, etc.) may provide a connection using wireless techniques, including digital cellular telephone connection, Cellular Digital Packet Data (CDPD) connection, digital satellite data connection, and/or another connection.

The signals associated with system 700 may include wireless communication signals such as radio frequency, electromagnetics, local area network (LAN), wide area network (WAN), virtual private network (VPN), wireless network (using 802.11, for example), 345 MHz, Z-WAVE®, cellular network (using 3G and/or LTE, for example), and/or other signals. The one or more antennas 735 and/or transceiver 730 may include or are related to, but are not limited to, WWAN (GSM, CDMA, and WCDMA), WLAN (including BLUETOOTH® and Wi-Fi), WMAN (WiMAX), antennas for mobile communications, antennas for Wireless Personal Area Network (WPAN) applications (including RFID and UWB). In some embodiments, each antenna 735 may receive signals or information specific and/or exclusive to itself. In other embodiments, each antenna 735 may receive signals or information not specific or exclusive to itself.

In some embodiments, one or more sensors 110-b may connect to some element of system 700 via a network using one or more wired and/or wireless connections (e.g., communication links 160). In some embodiments, the user interface 725 may include an audio device, such as an external speaker system, an external display device such as a display screen, and/or an input device (e.g., remote control device interfaced with the user interface 725 directly and/or through input/output controller 720). User interface may display data and enable interaction between data and a user as described with reference to FIG. 3. One or more buses 710 may allow data communication between one or more elements of apparatus 210-b (e.g., processor 705, memory 710, input/output controller 720, user interface 725, etc.).

The memory 710 may include random access memory (RAM), read only memory (ROM), flash RAM, and/or other types. The memory 710 may store computer-readable, computer-executable software/firmware code 715 including instructions that, when executed, cause the processor 705 to perform various functions described in this disclosure (e.g., cloud-based clinical logging). Alternatively, the software/firmware code 715 may not be directly executable by the processor 705 but may cause a computer (e.g., when compiled and executed) to perform functions described herein. Alternatively, the computer-readable, computer-executable software/firmware code 715 may not be directly executable by the processor 705 but may be configured to cause a computer (e.g., when compiled and executed) to perform functions described herein. The processor 705 may include an intelligent hardware device, e.g., a central processing unit (CPU), a microcontroller, an application-specific integrated circuit (ASIC), etc.

In some embodiments, the memory 710 may contain, among other things, the Basic Input-Output system (BIOS) which may control basic hardware and/or software operation such as the interaction with peripheral components or devices. Applications resident with system 700 are generally stored on and accessed via a non-transitory computer readable medium, such as a hard disk drive or other storage medium. Additionally, applications may be in the form of electronic signals modulated in accordance with the application and data communication technology when accessed via a network interface (e.g., transceiver 730, one or more antennas 735, etc.).

Many other devices and/or subsystems may be connected to one or more, or may be included as one or more elements of system 700 (e.g., medical devices, filters, entertainment systems, computing device, remote cameras, wireless key fob, wall mounted user interface devices, cell radio module, battery, alarm siren, door lock, lighting system, thermostat, home appliance monitor, utility equipment monitor, and so on). In some embodiments, all of the elements illustrated in FIG. 7 need not be present to practice the present systems and methods. The devices and subsystems may be interconnected in different ways from that illustrated in FIG. 7. In some embodiments, an aspect of some operation of a system, such as that illustrated in FIG. 7, may be readily known in the art and are not discussed in detail in this application. Code to implement the present disclosure may be stored in a non-transitory computer-readable medium such as one or more of system memory 710 or other memory. The operating system provided on input/output controller 720 may be iOS®, ANDROID®, MS-DOS®, MS-WINDOWS®, OS/2®, UNIX®, LINUX®, or another known operating system.

In some embodiments, the different components and modules described herein may be implemented as objects or processes that execute on a computing system (e.g., as separate threads). While some of the methods described herein are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely example representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the summary, detailed description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

The foregoing description, for purpose of explanation, has been described with reference to specific embodiments. However, the illustrative discussions above are not intended to be exhaustive or to limit the invention as claimed to the precise forms disclosed. Many modifications and variations are possible in view of the above teachings. The embodiments were chosen and described to explain practical applications, to thereby enable others skilled in the art to utilize the invention as claimed and various embodiments with various modifications as may be suited to the particular use contemplated.

1. A method for cloud-based clinical logging, the method comprising:
obtaining from a first medical device, by a processor, a first datum at a first time;
recording the first datum in a database; and
transmitting a first message to a first person regarding the first datum if the first datum does not satisfy a pre-determined threshold, where the first message requests an action based on the first datum not satisfying the pre-determined threshold.

2. The method of claim 1, wherein transmitting the first message further comprises:
transmitting a text message, e-mail, telephone call, instant message, or a combination thereof.

3. The method of claim 1, further comprising:
initiating an audio alarm, a visual alarm, or a combination thereof based on the first datum not satisfying the pre-determined threshold.

4. The method of claim 1, further comprising
determining that the requested action is not satisfied; and
transmitting a second message to a second person regarding the first message and the first datum based on determining that the requested action is not satisfied.

5. The method of claim 1, wherein determining that the first datum does not satisfy the pre-determined threshold further comprises:
obtaining a range of acceptable values for the first datum; and
determining that a value associated with the first datum is outside of the range of acceptable values.

6. The method of claim 1, further comprising:
determining a requested frequency for obtaining the first datum and at least a subsequent datum;
obtaining the subsequent datum based on the requested frequency; and
transmitting a third message to the first person regarding the subsequent datum if the subsequent datum does not satisfy the pre-determined threshold.

7. The method of claim 1, further comprising:
determining a time range between obtaining the first datum and at least a subsequent datum;
determining that the subsequent datum is not obtained within the time range; and
transmitting a fourth message to the first person regarding the subsequent datum if the subsequent datum is not obtained within the time range.

8. The method of claim 1, further comprising:
obtaining from the medical device, a second datum at a second time;
recording the second datum in the database;
determining a trend between the first datum and the second datum;
predicting failure of a component of the medical device based on the determined trend; and
requesting a second action based on predicting failure of the component.

9. The method of claim 8, wherein predicting failure of the component further comprises:
training a machine learning model on at least historical data obtained from the medical device, the first datum, and the second datum.

10. One or more non-transitory computer-readable media comprising one or more computer readable instructions, that when executed by one or more processors of a computing device, cause the computing device to perform a method for cloud-based data clinical logging, the method comprising:
obtaining from a first medical device, by a processor, a first datum at a first time;
recording the first datum in a database; and
transmitting a first message to a first person regarding the first datum if the first datum does not satisfy a pre-determined threshold, where the first message requests an action based on the first datum not satisfying the pre-determined threshold.

11. The non-transitory computer-readable media of claim 10, wherein transmitting the first message further comprises:
transmitting a text message, e-mail, telephone call, instant message, or a combination thereof.

12. The non-transitory computer-readable media of claim 10, further comprising:
initiating an audio alarm, a visual alarm, or a combination thereof based on the first datum not satisfying the pre-determined threshold.

13. The non-transitory computer-readable media of claim 10, further comprising:
determining that the requested action is not satisfied; and
transmitting a second message to a second person regarding the first message and the first datum based on determining that the requested action is not satisfied.

14. The non-transitory computer-readable media of claim 10, wherein determining that the first datum does not satisfy the pre-determined threshold further comprises:
obtaining a range of acceptable values for the first datum; and
determining that a value associated with the first datum is outside of the range of acceptable values.

15. The non-transitory computer-readable media of claim 10, further comprising:
determining a requested frequency for obtaining the first datum and at least a subsequent datum;
obtaining the subsequent datum based on the requested frequency; and
transmitting a third message to the first person regarding the subsequent datum if the subsequent datum does not satisfy the pre-determined threshold.

16. The non-transitory computer-readable media of claim 10, further comprising:
determining a time range between obtaining the first datum and at least a subsequent datum;
determining that the subsequent datum is not obtained within the time range; and
transmitting a fourth message to the first person regarding the subsequent datum if the subsequent datum is not obtained within the time range.

17. The non-transitory computer-readable media of claim 10, further comprising:
obtaining from the medical device, a second datum at a second time;
recording the second datum in the database;
determining a trend between the first datum and the second datum;
predicting failure of a component of the medical device based on the determined trend; and
requesting a second action based on predicting failure of the component.

18. The non-transitory computer-readable media of claim 17, wherein predicting failure of the component further comprises:
training a machine learning model on at least historical data obtained from the medical device, the first datum, and the second datum.

19. A computer device for cloud-based clinical logging, comprising:
a processor:
a memory in electronic communication with the processor:
instructions stored in the memory, the instructions being executable by the processor to:
obtain from a first medical device, by a processor, a first datum at a first time;
record the first datum in a database; and
transmit a first message to a first person regarding the first datum if the first datum does not satisfy a pre-determined threshold, where the first message requests an action based on the first datum not satisfying the pre-determined threshold.

20. The computer device of claim 18, wherein when the processor predicts failure of the component, the instructions are further executable to:
train a machine learning model on at least historical data obtained from the medical device, the first datum, and the second datum.
